(19) United States
(12) Patent Application Publication
(10) Pub. No.: US 2007/0269827 A1
(43) Pub. Date: Nov. 22, 2007

(54) Predicting and Diagnosing Patients With Autoimmune Disease

(75) Inventor: John B. Harley, Oklahoma City, OK (US)

Correspondence Address: FULBRIGHT & JAWORSKI L.L.P., 600 CONGRESS AVE., SUITE 2400, AUSTIN, TX, 78701, US

(73) Assignee: Oklahoma Medical Research Foundation

(21) Appl. No.: 11/750,729

(22) Filed: May 18, 2007

Related U.S. Application Data
(60) Provisional application No. 60/801,461, filed on May 18, 2006.
(60) Provisional application No. 60/868,513, filed on Dec. 4, 2006.

(51) Int. Cl. C12Q 1/68; C12P 19/34

(52) U.S. Cl. 435/6; 435/91.2

(57) ABSTRACT

The present invention provides methods for the prediction and diagnosis of autoimmune diseases including Systemic Lupus Erythematosus using a panel of single nucleotide polymorphisms (SNPs).

This application claims priority to U.S. Provisional Patent applications having Ser. No. 60/801,461 filed May 18, 2006 and Ser. No. 60/868,513 filed Dec. 4, 2006, both of which are incorporated herein by reference in their entirety.

### BACKGROUND OF THE INVENTION

A. Field of the Invention

The present invention relates to the fields of molecular biology, pathology and genetics. More specifically, the invention relates to methods of predicting and diagnosing automimmune disease based on the presence or absence of single nucleotide polymorphisms.

B. Related Art

Autoimmune diseases comprises a large number of widely varying illnesses. Their common feature is the existence of an immune response in the subject against one or more “self” antigens, including such wide ranging molecules as proteins, DNA and carbohydrates. These diseases can cause symptoms ranging from only mild discomfort to the patient, to complete debilitation and death. Most of autoimmune diseases remain very enigmatic, not only in their molecular basis and precipitating factors, but in their prediction, progression and treatment. As such, they continue to provide a considerable challenge to the healthcare industry.

Most genetic-based diseases do not generally have a simple, single genetic cause. Moreover, they are usually affected by environmental factors as well. The same can be said for autoimmune diseases, where defects in multiple genes often are involved. The situation is not aided by clinical diagnosis, since (a) familial autoimmune disease is often characterized by related individuals suffering from distinct autoimmune defects, and (b) the same autoimmune disease may manifest itself differently in different individuals at different times. Thus, one is left with a difficult, if not impossible, clinical diagnosis even when some genetic information is available. That is why researches continue to seek out better and more complete genetic bases for autoimmune diseases.

Systemic Lupus Erythematosus (SLE), like other autoimmune diseases, is mediated by a complex interaction of genetic and environmental elements. The genetic component of this interaction is clearly important: 20% of people with SLE have a relative who has or will have SLE. It is commonly believed that environmental factors may trigger a genetic predisposition to such diseases. Although the crucial role of genetic predisposition in susceptibility to SLE has been known for decades, only minimal progress has been made towards elucidating the specific genes involved in human disease. It is also suspected that SLE may be related to genetic defects in apoptosis. For example, mice lacking the gene for DNase1 develop SLE by 6 to 8 months of age.

Family studies have identified a number of genetic regions associated with elevated risk for SLE, although no specific genes have yet been identified. Harley et al. (1998); Wakeland et al. (2001). For example, 1q42 has been linked to SLE in three independent studies. Reviewed in Gaffney et al. (1998). Other genetic locations revealed by model-based linkage analysis include 1q23 and 11q 14 in African Americans, 14q11, 4p15, 11q25, 2q32, 19q 13, 6q26-27, and 12p 12-11 in European Americans, with 1q23, 13q32, 20q13, and 1q31 showing up in combined pedigrees. Moser et al. (1998). Associations have also been shown for the genetic markers HLA-DR2 and HLA-DR3. Arnett et al. (1992). More recently, expression profiling of peripheral blood mononuclear cells of SLE patients using microarrays has shown that about half of the patients demonstrate disregulated expression of genes in the IFN pathway. Baechler et al. (2003).

Despite these important observations, it is far from clear that one can predict the existence or predisposition to SLE based on this handful of genetic information. In all likelihood, a much more robust analysis using more and better genetic markers to identify SLE (and distinguish it from other autoimmune diseases) will be required.

### SUMMARY OF THE INVENTION

Thus, in accordance with the present invention, there is provided a method of identifying a subject afflicted with or at risk of developing an autoimmune disease comprising (a) obtaining a nucleic acid-containing sample from the subject; (b) analyzing a single nucleotide polymorphism (SNP) selected from those listed in Table X, wherein the presence of a SNP from Table X indicates that the subject is afflicted or at risk of developing an autoimmune disease. The method may further comprising analyzing a second, third, fourth, fifth SNP from Table X. Further SNPs from Table X may also be analyzed. The method may also further comprise analyzing a SNP from Table Z, which analysis may also be extended to a second, third, fourth or fifth SNP from Table Z. The method may further comprise treating the subject based on the results of step (b). The method may further comprise taking a clinical history from the subject. Analysis may comprises nucleic acid amplification, such as PCR. Analysis may also comprise primer extension, restriction digestion, sequencing, SNP specific oligonucleotide hybridization, or a DNAse protection assay. The sample may be blood, sputum, saliva, mucosal scraping or tissue biopsy.

The autoimmune disease may be systemic lupus erythematosus, Sjogren's syndrome, rheumatoid arthritis, juvenile onset diabetes mellitus, Wegener's granulomatosis, inflammatory bowel disease, polymyositis, dermatomyositis, multiple endocrine failure, Schmidt's syndrome, autoimmune uveitis, Addison's disease, adrenalitis, Graves' disease, thyroiditis, Hashimoto's thyroiditis, autoimmune thyroid disease, pernicious anemia, gastric atrophy, chronic hepatitis, lupoid hepatitis, atherosclerosis, presenile dementia, demyelinating diseases, multiple sclerosis, subacute cutaneous lupus erythematosus, hypoparathyroidism, Dressler's syndrome, myasthenia gravis, autoimmune thrombocytopenia, idiopathic thrombocytopenic purpura, hemolytic anemia, pemphigus vulgaris, pemphigus, dermatitis herpetiformis, alopecia arcata, pemphigoid, scleroderma, progressive systemic sclerosis, CREST syndrome (calcinosis, Raynaud's phenomenon, esophageal dysmotility, sclerodactyly, and telangiectasia), adult onset diabetes mellitus (Type II diabetes), male and female autoimmune infertility, ankylosing spondolytis, ulcerative colitis, Crohn's disease, mixed connective tissue disease, polyarteritis nedosa, systemic necrotizing vasculitis, juvenile onset rheumatoid arthritis, glomerulonephritis, atopic dermatitis, atopic rhinitis, Goodpasture's syndrome, Chagas' disease, sarcoidosis, rheumatic fever, asthma, recurrent abortion, anti-phospholipid syndrome, farmer's lung, erythema multiforme, post cardiotomy syndrome, Cushing's syndrome, autoimmune chronic active hepatitis, bird-fancier's lung, allergic disease, allergic encephalomyelitis, toxic epidermal necrolysis, alopecia, Alport's syndrome, alveolitis, allergic alveolitis, fibrosing alveolitis, interstitial lung disease, erythema nodosum, pyoderma gangrenosum, transfusion reaction, leprosy, malaria, leishmaniasis, trypanosomiasis, Takayasu's arteritis, polymyalgia rheumatica, temporal arteritis, schistosomiasis, giant cell arteritis, ascariasis, aspergillosis, Sampter's syndrome, eczema, lymphomatoid granulomatosis, Behcet's disease, Caplan's syndrome, Kawasaki's disease, dengue, encephalomyelitis, endocarditis, endomyocardial fibrosis, endophthalmitis, erythema elevatum et diutinum, psoriasis, erythroblastosis fetalis, eosinophilic faciitis, Shulman's syndrome, Felty's syndrome, filariasis, cyclitis, chronic cyclitis, heterochronic cyclitis, Fuch's cyclitis, IgA nephropathy, Henoch-Schonlein purpura, glomerulonephritis, graft versus host disease, transplantation rejection, human immunodeficiency virus infection, echovirus infection, cardiomyopathy, Alzheimer's disease, parvovirus infection, rubella virus infection, post vaccination syndromes, congenital rubella infection, Hodgkin's and Non-Hodgkin's lymphoma, renal cell carcinoma, multiple myeloma, Eaton-Lambert syndrome, relapsing polychondritis, malignant melanoma, cryoglobulinemia, Waldenstrom's macroglobulemia, Epstein-Barr virus infection, mumps, Evan's syndrome, or autoimmune gonadal failure.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.”

It is contemplated that any embodiment discussed in this specification can be implemented with respect to any method or composition of the invention, and vice versa. Furthermore, compositions and kits of the invention can be used to achieve methods of the invention.

Throughout this application, the term “about” is used to indicate that a value includes the inherent variation of error for the device, the method being employed to determine the value, or the variation that exists among the study subjects.

### DETAILED DESCRIPTION OF THE INVENTION

### I. SNP-Based Diagnostics

Knowledge of DNA polymorphisms can prove very useful in a variety of applications, including diagnosis and treatment of autoimmune disease. A particular kind of polymorphism, called a single nucleotide polymorphism, or SNP (pronounced “snip”), is a small genetic change or variation that can occur within a person's DNA sequence. The genetic code is specified by the four nucleotide “letters” A (adenine), C (cytosine), T (thymine), and G (guanine). SNP variation occurs when a single nucleotide, such as an A, replaces one of the other three nucleotide letters—C, G, or T.

An example of a SNP is the alteration of the DNA segment AAGGTTA to ATGGTTA, where the second “A” in the first snippet is replaced with a “T.” On average, SNPs occur in the human population more than 1 percent of the time. Because only about 3 to 5 percent of a person's DNA sequence codes for the production of proteins, most SNPs are found outside of “coding sequences.” SNPs found within a coding sequence are of particular interest to researchers because they are more likely to alter the biological function of a protein. Because of the recent advances in technology, coupled with the unique ability of these genetic variations to facilitate gene identification, there has been a recent flurry of SNP discovery and detection.

Finding single nucleotide changes in the human genome seems like a daunting prospect, but over the last 20 years, biomedical researchers have developed a number of techniques that make it possible to do just that. Each technique uses a different method to compare selected regions of a DNA sequence obtained from multiple individuals who share a common trait. In each test, the result shows a physical difference in the DNA samples only when a SNP is detected in one individual and not in the other.

Many common diseases in humans are not caused by a genetic variation within a single gene, but instead are influenced by complex interactions among multiple genes as well as environmental and lifestyle factors. Although both environmental and lifestyle factors add tremendously to the uncertainty of developing a disease, it is currently difficult to measure and evaluate their overall effect on a disease process. Therefore, when looking at SNPs, one refers mainly to a person's genetic predisposition, or the potential of an individual to develop a disease based on genes and hereditary factors. This is particularly true in diagnosis of autoimmune disease.

Each person's genetic material contains a unique SNP pattern that is made up of many different genetic variations. Researchers have found that most SNPs are not responsible for a disease state. Instead, they serve as biological markers for pinpointing a disease on the human genome map, because they are usually located near a gene found to be associated with a certain disease. Occasionally, a SNP may actually cause a disease and, therefore, can be used to search for and isolate the disease-causing gene.

To create a genetic test that will screen for an autoimmune disease, one will collect blood or tissue samples from a group of individuals affected by the disease and analyze their DNA for SNP patterns. One then compares these patterns to patterns obtained by analyzing the DNA from a group of individuals unaffected by the disease. This type of comparison, called an “association study,” can detect differences between the SNP patterns of the two groups, thereby indicating which pattern is most likely associated with the disease-causing gene. Eventually, SNP profiles that are characteristic of a variety of diseases will be established. These profiles can then be applied to the population at general, or those deemed to be at particular risk of developing an autoimmune disease.

A. Methods of Assaying for SNPs

There are a large variety of techniques that can be used to assess SNPs, and more are being discovered each day. The following is a very general discussion of a few of these techniques that can be used in accordance with the present invention.

1. RFLP

Restriction Fragment Length Polymorphism (RFLP) is a technique in which different DNA sequences may be differentiated by analysis of patterns derived from cleavage of that DNA. If two sequences differ in the distance between sites of cleavage of a particular restriction endonuclease, the length of the fragments produced will differ when the DNA is digested with a restriction enzyme. The similarity of the patterns generated can be used to differentiate species (and even strains) from one another.

Restriction endonucleases in turn are the enzymes that cleave DNA molecules at specific nucleotide sequences depending on the particular enzyme used. Enzyme recognition sites are usually 4 to 6 base pairs in length. Generally, the shorter the recognition sequence, the greater the number of fragments generated. If molecules differ in nucleotide sequence, fragments of different sizes may be generated. The fragments can be separated by gel electrophoresis. Restriction enzymes are isolated from a wide variety of bacterial genera and are thought to be part of the cell's defenses against invading bacterial viruses. Use of RFLP and restriction endonucleases in SNP analysis requires that the SNP affect cleavage of at least one restriction enzyme site.

2. Primer Extension

The primer and no more than three NTPs may be combined with a polymerase and the target sequence, which serves as a template for amplification. By using less than all four NTPs, it is possible to omit one or more of the polymorphic nucleotides needed for incorporation at the polymorphic site. It is important for the practice of the present invention that the amplification be designed such that the omitted nucleotide(s) is(are) not required between the 3′ end of the primer and the target polymorphism. The primer is then extended by a nucleic acid polymerase, in a preferred embodiment by Taq polymerase. If the omitted NTP is required at the polymorphic site, the primer is extended up to the polymorphic site, at which point the polymerization ceases. However, if the omitted NTP is not required at the polymorphic site, the primer will be extended beyond the polymorphic site, creating a longer product. Detection of the extension products is based on, for example, separation by size/length which will thereby reveal which polymorphism is present.

A specific form of primer extension, developed by the inventor, can be found in U.S. Ser. No. 10/407,846, which is hereby specifically incorporated by reference.

3. Oligonucleotide Hybridization

Oligonucleotides may be designed to hybridize directly to a target site of interest. The most common form of such analysis is where oligonucleotides are arrayed on a chip or plate in a “microarray.” Microarrays comprise a plurality of oligos spatially distributed over, and stably associated with, the surface of a substantially planar substrate, e.g., biochips. Microarrays of oligonucleotides have been developed and find use in a variety of applications, such as screening and DNA sequencing.

In gene analysis with microarrays, an array of “probe” oligonucleotides is contacted with a nucleic acid sample of interest, i.e., target. Contact is carried out under hybridization conditions and unbound nucleic acid is then removed. The resultant pattern of hybridized nucleic acid provides information regarding the genetic profile of the sample tested. Methodologies of gene analysis on microarrays are capable of providing both qualitative and quantitative information.

A variety of different arrays which may be used are known in the art. The probe molecules of the arrays which are capable of sequence specific hybridization with target nucleic acid may be polynucleotides or hybridizing analogues or mimetics thereof, including: nucleic acids in which the phosphodiester linkage has been replaced with a substitute linkage, such as phophorothioate, methylimino, methylphosphonate, phosphoramidate, guanidine and the like; nucleic acids in which the ribose subunit has been substituted, e.g., hexose phosphodiester; peptide nucleic acids; and the like. The length of the probes will generally range from 10 to 1000 nts, where in some embodiments the probes will be oligonucleotides and usually range from 15 to 150 nts and more usually from 15 to 100 nts in length, and in other embodiments the probes will be longer, usually ranging in length from 150 to 1000 nts, where the polynucleotide probes may be single- or double-stranded, usually single-stranded, and may be PCR fragments amplified from cDNA.

The probe molecules on the surface of the substrates will correspond to selected genes being analyzed and be positioned on the array at a known location so that positive hybridization events may be correlated to expression of a particular gene in the physiological source from which the target nucleic acid sample is derived. The substrates with which the probe molecules are stably associated may be fabricated from a variety of materials, including plastics, ceramics, metals, gels, membranes, glasses, and the like. The arrays may be produced according to any convenient methodology, such as preforming the probes and then stably associating them with the surface of the support or growing the probes directly on the support. A number of different array configurations and methods for their production are known to those of skill in the art and disclosed in U.S. Pat. Nos. 5,445,934, 5,532,128, 5,556,752, 5,242,974, 5,384,261, 5,405,783, 5,412,087, 5,424,186, 5,429,807, 5,436,327, 5,472,672, 5,527,681, 5,529,756, 5,545,531, 5,554,501, 5,561,071, 5,571,639, 5,593,839, 5,599,695, 5,624,711, 5,658,734, 5,700,637, and 6,004,755.

Following hybridization, where non-hybridized labeled nucleic acid is capable of emitting a signal during the detection step, a washing step is employed where unhybridized labeled nucleic acid is removed from the support surface, generating a pattern of hybridized nucleic acid on the substrate surface. A variety of wash solutions and protocols for their use are known to those of skill in the art and may be used.

Where the label on the target nucleic acid is not directly detectable, one then contacts the array, now comprising bound target, with the other member(s) of the signal producing system that is being employed. For example, where the label on the target is biotin, one then contacts the array with streptavidin-fluorescer conjugate under conditions sufficient for binding between the specific binding member pairs to occur. Following contact, any unbound members of the signal producing system will then be removed, e.g., by washing. The specific wash conditions employed will necessarily depend on the specific nature of the signal producing system that is employed, and will be known to those of skill in the art familiar with the particular signal producing system employed.

The resultant hybridization pattern(s) of labeled nucleic acids may be visualized or detected in a variety of ways, with the particular manner of detection being chosen based on the particular label of the nucleic acid, where representative detection means include scintillation counting, autoradiography, fluorescence measurement, calorimetric measurement, light emission measurement and the like.

Prior to detection or visualization, where one desires to reduce the potential for a mismatch hybridization event to generate a false positive signal on the pattern, the array of hybridized target/probe complexes may be treated with an endonuclease under conditions sufficient such that the endonuclease degrades single stranded, but not double stranded DNA. A variety of different endonucleases are known and may be used, where such nucleases include: mung bean nuclease, S1 nuclease, and the like. Where such treatment is employed in an assay in which the target nucleic acids are not labeled with a directly detectable label, e.g., in an assay with biotinylated target nucleic acids, the endonuclease treatment will generally be performed prior to contact of the array with the other member(s) of the signal producing system, e.g., fluorescent-streptavidin conjugate. Endonuclease treatment, as described above, ensures that only end-labeled target/probe complexes having a substantially complete hybridization at the 3′ end of the probe are detected in the hybridization pattern.

Following hybridization and any washing step(s) and/or subsequent treatments, as described above, the resultant hybridization pattern is detected. In detecting or visualizing the hybridization pattern, the intensity or signal value of the label will be not only be detected but quantified, by which is meant that the signal from each spot of the hybridization will be measured and compared to a unit value corresponding the signal emitted by known number of end-labeled target nucleic acids to obtain a count or absolute value of the copy number of each end-labeled target that is hybridized to a particular spot on the array in the hybridization pattern.

4. Sequencing

DNA sequencing enables one to perform a thorough analysis of DNA because it provides the most basic information of all: the sequence of nucleotides. Maxam & Gilbert developed the first widely used sequencing methods—a “chemical cleavage protocol.” Shortly thereafter, Sanger designed a procedure similar to the natural process of DNA replication. Even though both teams shared the 1980 Nobel Prize, Sanger's method became the standard because of its practicality.

Sanger's method, which is also referred to as dideoxy sequencing or chain termination, is based on the use of dideoxynucleotides (ddNTP's) in addition to the normal nucleotides (NTP's) found in DNA. Dideoxynucleotides are essentially the same as nucleotides except they contain a hydrogen group on the 3′ carbon instead of a hydroxyl group (OH). These modified nucleotides, when integrated into a sequence, prevent the addition of further nucleotides. This occurs because a phosphodiester bond cannot form between the dideoxynucleotide and the next incoming nucleotide, and thus the DNA chain is terminated. Using this method, optionally coupled with amplification of the nucleic acid target, one can now rapidly sequence large numbers of target molecules, usually employing automated sequencing apparati. Such techniques are well known to those of skill in the art.

B. Detection Systems

1. Mass Spectromety

By exploiting the intrinsic properties of mass and charge, mass spectrometry (MS) can resolved and confidently identified a wide variety of complex compounds. Traditional quantitative MS has used electrospray ionization (ESI) followed by tandem MS (MS/MS) (Chen et al., 2001; Zhong et al., 2001; Wu et al., 2000) while newer quantitative methods are being developed using matrix assisted laser desorption/ionization (MALDI) followed by time of flight (TOF) MS (Bucknall et al., 2002; Mirgorodskaya et al., 2000; Gobom et al., 2000).

i. ESI

ESI is a convenient ionization technique developed by Fenn and colleagues (Fenn et al., 1989) that is used to produce gaseous ions from highly polar, mostly nonvolatile biomolecules, including lipids. The sample is injected as a liquid at low flow rates (1-10 μL/min) through a capillary tube to which a strong electric field is applied. The field generates additional charges to the liquid at the end of the capillary and produces a fine spray of highly charged droplets that are electrostatically attracted to the mass spectrometer inlet. The evaporation of the solvent from the surface of a droplet as it travels through the desolvation chamber increases its charge density substantially. When this increase exceeds the Rayleigh stability limit, ions are ejected and ready for MS analysis.

A typical conventional ESI source consists of a metal capillary of typically 0.1-0.3 mm in diameter, with a tip held approximately 0.5 to 5 cm (but more usually 1 to 3 cm) away from an electrically grounded circular interface having at its center the sampling orifice, such as described by Kabarle et al. (1993). A potential difference of between 1 to 5 kV (but more typically 2 to 3 kV) is applied to the capillary by power supply to generate a high electrostatic field (10⁶ to 10⁷ V/m) at the capillary tip. A sample liquid carrying the analyte to be analyzed by the mass spectrometer, is delivered to tip through an internal passage from a suitable source (such as from a chromatograph or directly from a sample solution via a liquid flow controller). By applying pressure to the sample in the capillary, the liquid leaves the capillary tip as a small highly electrically charged droplets and further undergoes desolvation and breakdown to form single or multi-charged gas phase ions in the form of an ion beam. The ions are then collected by the grounded (or negatively-charged) interface plate and led through an the orifice into an analyzer of the mass spectrometer. During this operation, the voltage applied to the capillary is held constant. Aspects of construction of ESI sources are described, for example, in U.S. Pat. Nos. 5,838,002; 5,788,166; 5,757,994; RE 35,413; and 5,986,258.

ii. ESI/MS/MS

In ESI tandem mass spectroscopy (ESI/MS/MS), one is able to simultaneously analyze both precursor ions and product ions, thereby monitoring a single precursor product reaction and producing (through selective reaction monitoring (SRM)) a signal only when the desired precursor ion is present. When the internal standard is a stable isotope-labeled version of the analyte, this is known as quantification by the stable isotope dilution method. This approach has been used to accurately measure pharmaceuticals (Zweigenbaum et al., 2000; Zweigenbaum et al., 1999) and bioactive peptides (Desiderio et al., 1996; Lovelace et al., 1991). Newer methods are performed on widely available MALDI-TOF instruments, which can resolve a wider mass range and have been used to quantify metabolites, peptides, and proteins. Larger molecules such as peptides can be quantified using unlabeled homologous peptides as long as their chemistry is similar to the analyte peptide (Duncan et al., 1993; Bucknall et al., 2002). Protein quantification has been achieved by quantifying tryptic peptides (Mirgorodskaya et al., 2000). Complex mixtures such as crude extracts can be analyzed, but in some instances sample clean up is required (Nelson et al., 1994; Gobom et al., 2000).

iii. SIMS

Secondary ion mass spectroscopy, or SIMS, is an analytical method that uses ionized particles emitted from a surface for mass spectroscopy at a sensitivity of detection of a few parts per billion. The sample surface is bombarded by primary energetic particles, such as electrons, ions (e.g., O, Cs), neutrals or even photons, forcing atomic and molecular particles to be ejected from the surface, a process called sputtering. Since some of these sputtered particles carry a charge, a mass spectrometer can be used to measure their mass and charge. Continued sputtering permits measuring of the exposed elements as material is removed. This in turn permits one to construct elemental depth profiles. Although the majority of secondary ionized particles are electrons, it is the secondary ions which are detected and analysis by the mass spectrometer in this method.

iv. LD-MS and LDLPMS

Laser desorption mass spectroscopy (LD-MS) involves the use of a pulsed laser, which induces desorption of sample material from a sample site—effectively, this means vaporization of sample off of the sample substrate. This method is usually only used in conjunction with a mass spectrometer, and can be performed simultaneously with ionization if one uses the right laser radiation wavelength.

When coupled with Time-of-Flight (TOF) measurement, LD-MS is referred to as LDLPMS (Laser Desorption Laser Photoionization Mass Spectroscopy). The LDLPMS method of analysis gives instantaneous volatilization of the sample, and this form of sample fragmentation permits rapid analysis without any wet extraction chemistry. The LDLPMS instrumentation provides a profile of the species present while the retention time is low and the sample size is small. In LDLPMS, an impactor strip is loaded into a vacuum chamber. The pulsed laser is fired upon a certain spot of the sample site, and species present are desorbed and ionized by the laser radiation. This ionization also causes the molecules to break up into smaller fragment-ions. The positive or negative ions made are then accelerated into the flight tube, being detected at the end by a microchannel plate detector. Signal intensity, or peak height, is measured as a function of travel time. The applied voltage and charge of the particular ion determines the kinetic energy, and separation of fragments are due to different size causing different velocity. Each ion mass will thus have a different flight-time to the detector.

One can either form positive ions or negative ions for analysis. Positive ions are made from regular direct photoionization, but negative ion formation require a higher powered laser and a secondary process to gain electrons. Most of the molecules that come off the sample site are neutrals, and thus can attract electrons based on their electron affinity. The negative ion formation process is less efficient than forming just positive ions. The sample constituents will also affect the outlook of a negative ion spectra.

Other advantages with the LDLPMS method include the possibility of constructing the system to give a quiet baseline of the spectra because one can prevent coevolved neutrals from entering the flight tube by operating the instrument in a linear mode. Also, in environmental analysis, the salts in the air and as deposits will not interfere with the laser desorption and ionization. This instrumentation also is very sensitive, known to detect trace levels in natural samples without any prior extraction preparations.

v. MALDI-TOF-MS

Since its inception and commercial availability, the versatility of MALDI-TOF-MS has been demonstrated convincingly by its extensive use for qualitative analysis. For example, MALDI-TOF-MS has been employed for the characterization of synthetic polymers (Marie et al., 2000; Wu et al., 1998). peptide and protein analysis (Roepstorff et al., 2000; Nguyen et al., 1995), DNA and oligonucleotide sequencing (Miketova et al., 1997; Faulstich et al., 1997; Bentzley et al., 1996), and the characterization of recombinant proteins (Kanazawa et al., 1999; Villanueva et al., 1999). Recently, applications of MALDI-TOF-MS have been extended to include the direct analysis of biological tissues and single cell organisms with the aim of characterizing endogenous peptide and protein constituents (Li et al., 2000; Lynn et al., 1999; Stoeckli et al., 2001; Caprioli et al., 1997; Chaurand et al., 1999; Jespersen et al., 1999).

The properties that make MALDI-TOF-MS a popular qualitative tool-its ability to analyze molecules across an extensive mass range, high sensitivity, minimal sample preparation and rapid analysis times—also make it a potentially useful quantitative tool. MALDI-TOF-MS also enables non-volatile and thermally labile molecules to be analyzed with relative ease. It is therefore prudent to explore the potential of MALDI-TOF-MS for quantitative analysis in clinical settings, for toxicological screenings, as well as for environmental analysis. In addition, the application of MALDI-TOF-MS to the quantification of peptides and proteins is particularly relevant. The ability to quantify intact proteins in biological tissue and fluids presents a particular challenge in the expanding area of proteomics and investigators urgently require methods to accurately measure the absolute quantity of proteins. While there have been reports of quantitative MALDI-TOF-MS applications, there are many problems inherent to the MALDI ionization process that have restricted its widespread use (Kazmaier et al., 1998; Horak et al., 2001; Gobom et al., 2000; Wang et al., 2000; Desiderio et al., 2000). These limitations primarily stem from factors such as the sample/matrix heterogeneity, which are believed to contribute to the large variability in observed signal intensities for analytes, the limited dynamic range due to detector saturation, and difficulties associated with coupling MALDI-TOF-MS to on-line separation techniques such as liquid chromatography. Combined, these factors are thought to compromise the accuracy, precision, and utility with which quantitative determinations can be made.

Because of these difficulties, practical examples of quantitative applications of MALDI-TOF-MS have been limited. Most of the studies to date have focused on the quantification of low mass analytes, in particular, alkaloids or active ingredients in agricultural or food products (Wang et al., 1999; Jiang et al., 2000; Wang et al., 2000; Yang et al., 2000; Wittmann et al., 2001), whereas other studies have demonstrated the potential of MALDI-TOF-MS for the quantification of biologically relevant analytes such as neuropeptides, proteins, antibiotics, or various metabolites in biological tissue or fluid (Muddiman et al., 1996; Nelson et al., 1994; Duncan et al., 1993; Gobom et al., 2000; Wu et al., 1997; Mirgorodskaya et al., 2000). In earlier work it was shown that linear calibration curves could be generated by MALDI-TOF-MS provided that an appropriate internal standard was employed (Duncan et al., 1993). This standard can “correct” for both sample-to-sample and shot-to-shot variability. Stable isotope labeled internal standards (isotopomers) give the best result.

With the marked improvement in resolution available on modern commercial instruments, primarily because of delayed extraction (Bahr et al., 1997; Takach et al., 1997), the opportunity to extend quantitative work to other examples is now possible; not only of low mass analytes, but also biopolymers. Of particular interest is the prospect of absolute multi-component quantification in biological samples (e.g., proteomics applications).

The properties of the matrix material used in the MALDI method are critical. Only a select group of compounds is useful for the selective desorption of proteins and polypeptides. A review of all the matrix materials available for peptides and proteins shows that there are certain characteristics the compounds must share to be analytically useful. Despite its importance, very little is known about what makes a matrix material “successful” for MALDI. The few materials that do work well are used heavily by all MALDI practitioners and new molecules are constantly being evaluated as potential matrix candidates. With a few exceptions, most of the matrix materials used are solid organic acids. Liquid matrices have also been investigated, but are not used routinely.

2. Hybridization

There are a variety of ways by which one can assess genetic profiles, and may of these rely on nucleic acid hybridization. Hybridization is defined as the ability of a nucleic acid to selectively form duplex molecules with complementary stretches of DNAs and/or RNAs. Depending on the application envisioned, one would employ varying conditions of hybridization to achieve varying degrees of selectivity of the probe or primers for the target sequence.

Typically, a probe or primer of between 13 and 100 nucleotides, preferably between 17 and 100 nucleotides in length up to 1-2 kilobases or more in length will allow the formation of a duplex molecule that is both stable and selective. Molecules having complementary sequences over contiguous stretches greater than 20 bases in length are generally preferred, to increase stability and selectivity of the hybrid molecules obtained. One will generally prefer to design nucleic acid molecules for hybridization having one or more complementary sequences of 20 to 30 nucleotides, or even longer where desired. Such fragments may be readily prepared, for example, by directly synthesizing the fragment by chemical means or by introducing selected sequences into recombinant vectors for recombinant production.

For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting specific mRNA transcripts. It is generally appreciated that conditions can be rendered more stringent by the addition of increasing amounts of formamide.

For certain applications, for example, lower stringency conditions may be used. Under these conditions, hybridization may occur even though the sequences of the hybridizing strands are not perfectly complementary, but are mismatched at one or more positions. Conditions may be rendered less stringent by increasing salt concentration and/or decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Hybridization conditions can be readily manipulated depending on the desired results.

In other embodiments, hybridization may be achieved under conditions of, for example, 50 mM Tris-HCl (pH 8.3), 75 mM KCl, 3 mM MgCl₂, 1.0 mM dithiothreitol, at temperatures between approximately 20° C. to about 37° C. Other hybridization conditions utilized could include approximately 10 mM Tris-HCl (pH 8.3), 50 mM KCl, 1.5 mM MgCl₂, at temperatures ranging from approximately 40° C. to about 72° C.

In certain embodiments, it will be advantageous to employ nucleic acids of defined sequences of the present invention in combination with an appropriate means, such as a label, for determining hybridization. A wide variety of appropriate indicator means are known in the art, including fluorescent, radioactive, enzymatic or other ligands, such as avidin/biotin, which are capable of being detected. In preferred embodiments, one may desire to employ a fluorescent label or an enzyme tag such as urease, alkaline phosphatase or peroxidase, instead of radioactive or other environmentally undesirable reagents. In the case of enzyme tags, colorimetric indicator substrates are known that can be employed to provide a detection means that is visibly or spectrophotometrically detectable, to identify specific hybridization with complementary nucleic acid containing samples.

In general, it is envisioned that the probes or primers described herein will be useful as reagents in solution hybridization, as in PCR™, for detection of expression of corresponding genes, as well as in embodiments employing a solid phase. In embodiments involving a solid phase, the test DNA (or RNA) is adsorbed or otherwise affixed to a selected matrix or surface. This fixed, single-stranded nucleic acid is then subjected to hybridization with selected probes under desired conditions. The conditions selected will depend on the particular circumstances (depending, for example, on the G+C content, type of target nucleic acid, source of nucleic acid, size of hybridization probe, etc.). Optimization of hybridization conditions for the particular application of interest is well known to those of skill in the art. After washing of the hybridized molecules to remove non-specifically bound probe molecules, hybridization is detected, and/or quantified, by determining the amount of bound label. Representative solid phase hybridization methods are disclosed in U.S. Pat. Nos. 5,843,663, 5,900,481 and 5,919,626. Other methods of hybridization that may be used in the practice of the present invention are disclosed in U.S. Pat. Nos. 5,849,481, 5,849,486 and 5,851,772. The relevant portions of these and other references identified in this section of the Specification are incorporated herein by reference.

3. Detectable Labels

Various nucleic acids may be visualized in order to confirm their presence, quantity or sequence. In one embodiment, the primer is conjugated to a chromophore but may instead be radiolabeled or fluorometrically labeled. In another embodiment, the primer is conjugated to a binding partner that carries a detectable moiety, such as an antibody or biotin. In other embodiments, the primer incorporates a fluorescent dye or label. In yet other embodiments, the primer has a mass label that can be used to detect the molecule amplified. Other embodiments also contemplate the use of Taqman™ and Molecular Beacon™ probes. Alternatively, one or more of the dNTPs may be labeled with a radioisotope, a fluorophore, a chromophore, a dye or an enzyme. Also, chemicals whose properties change in the presence of DNA can be used for detection purposes. For example, the methods may involve staining of a gel with, or incorporation into the separation media, a fluorescent dye, such as ethidium bromide or Vistra Green, and visualization under an appropriate light source.

The choice of label incorporated into the products is dictated by the method used for analysis. When using capillary electrophoresis, microfluidic electrophoresis, HPLC, or LC separations, either incorporated or intercalated fluorescent dyes are used to label and detect the amplification products. Samples are detected dynamically, in that fluorescence is quantitated as a labeled species moves past the detector. If any electrophoretic method, HPLC, or LC is used for separation, products can be detected by absorption of UV light, a property inherent to DNA and therefore not requiring addition of a label. If polyacrylamide gel or slab gel electrophoresis is used, the primer for the extension reaction can be labeled with a fluorophore, a chromophore or a radioisotope, or by associated enzymatic reaction. Alternatively, if polyacrylamide gel or slab gel electrophoresis is used, one or more of the NTPs in the extension reaction can be labeled with a fluorophore, a chromophore or a radioisotope, or by associated enzymatic reaction. Enzymatic detection involves binding an enzyme to a nucleic acid, e.g., via a biotin:avidin interaction, following separation of the amplification products on a gel, then detection by chemical reaction, such as chemiluminescence generated with luminol. A fluorescent signal can be monitored dynamically. Detection with a radioisotope or enzymatic reaction requires an initial separation by gel electrophoresis, followed by transfer of DNA molecules to a solid support (blot) prior to analysis. If blots are made, they can be analyzed more than once by probing, stripping the blot, and then reprobing. If the extension products are separated using a mass spectrometer no label is required because nucleic acids are detected directly.

In the case of radioactive isotopes, tritium, ¹⁴C and ³²P are used predominantly. Among the fluorescent labels contemplated for use as conjugates include Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, TAMRA, TET, Tetramethylrhodamine, and/or Texas Red.

4. Other Methods of Detecting Nucleic Acids

Other methods of nucleic acid detection that may be used in the practice of the instant invention are disclosed in U.S. Pat. Nos. 5,840,873, 5,843,640, 5,843,651, 5,846,708, 5,846,717, 5,846,726, 5,846,729, 5,849,487, 5,853,990, 5,853,992, 5,853,993, 5,856,092, 5,861,244, 5,863,732, 5,863,753, 5,866,331, 5,905,024, 5,910,407, 5,912,124, 5,912,145, 5,919,630, 5,925,517, 5,928,862, 5,928,869, 5,929,227, 5,932,413 and 5,935,791, each of which is incorporated herein by reference in its entirety.

5. Selection and of Primers/Probes/Enzymes

The present invention relies on the use of agents that are capable of detecting single nucleotide changes in DNA. These agents generally fall into two classes—agents that hybridize to target sequences that contain the change, and agents that hybridize to target sequences that are adjacent to (e.g., upstream or 5′ to) the region of change. A third class of agents, restriction enzymes, do not hybridize, but instead cleave at a target site. A list of restriction enzymes can be found at www.fermentas.com/techinfo/re/prototypes.htm, hereby incorporated by reference.

The present invention relies up the identification of SNPs from Table X that have association with autoimmune disease. The reference numbers provided for these SNPs are from the NCBI SNP database, at www.ncbi.nlm.nih.gov/entrez/query.fcgi?db=snp, the relevant portions of which are hereby incorporated by reference. Thus, one will select and design probes/primers, depending on what technique will be used to interrogate the DNA of interest. That probe may either hybridize to a target sequence or adjacent to that sequence.

TABLE X

| | | | |
|---|---|---|---|
| | | BUILD | |
| SNPNAME | GENE | 35 POSITION | CHROMOSOME |

| | | | |
|---|---|---|---|
| rs2004640 | IRF5 | 1.28E+08 | 7 |
| rs3732630 | DNASE1L3 | 58154068 | 3 |
| rs3807306 | IRF5 | 1.28E+08 | 7 |
| rs1805010 | IL4R | 27263704 | 16 |
| rs10515227 | FLJ25333 | 94824898 | 5 |
| rs1017643 | LOC389437 | 1.57E+08 | 6 |
| rs4234743 | PPP2R2C | 6593877 | 4 |
| rs751609 | CAPN2 | 2.2E+08 | 1 |
| rs1017528 | CUEDC1 | 53367550 | 17 |
| rs4484301 | TBC1D14 | 7073994 | 4 |
| rs905010 | SORCS2 | 7371332 | 4 |
| rs5361 | SELE | 1.66E+08 | 1 |
| rs3746250 | CD22 | 40527422 | 19 |
| rs1874328 | IRF5 | 1.28E+08 | 7 |
| rs493644 | LOC400792 | 1.58E+08 | 1 |
| rs4784214 | TNRC9 | 51044314 | 16 |
| rs2267574 | CD22 | 40516983 | 19 |
| rs1423380 | SIAT8D | 1E+08 | 5 |
| rs3776176 | SIAT8D | 1E+08 | 5 |
| rs2280714 | TNPO3 | 1.28E+08 | 7 |
| rs10515290 | Null | 99277365 | 5 |
| rs1788242 | CD226 | 65696958 | 18 |
| rs17841953 | HLA-DRB1 | 32595290 | 6 |
| rs8057341 | CARD15 | 49295481 | 16 |
| rs1268524 | SLAMF6 | 1.57E+08 | 1 |
| rs2230748 | CD97 | 14373489 | 19 |
| rs2057768 | IL4R | 27229596 | 16 |
| rs271653 | LOC123865 | 45360879 | 17 |
| rs1523383 | Null | 98727933 | 5 |
| rs891779 | CAMKK2 | 1.2E+08 | 12 |
| rs7755898 | CYP21A1P | 32083441 | 6 |
| rs4331842 | PPP2R2C | 6599853 | 4 |
| rs8048583 | ITGAM | 31187037 | 16 |
| rs2069949 | PROCR | 33226943 | 20 |
| rs2049051 | RAB3A | 18176831 | 19 |
| rs2291739 | TIMELESS | 55100920 | 12 |
| rs3741240 | SCGB1A1 | 61943118 | 11 |
| rs2683053 | CYP4F3 | 15627470 | 19 |
| rs3894735 | SORCS2 | 7477178 | 4 |
| rs10937735 | PPP2R2C | 6589318 | 4 |
| rs2107356 | IL4R | 27230905 | 16 |
| rs161507 | Null | 99675570 | 5 |
| rs118003 | null | 2.1E+08 | 1 |
| rs3822616 | KIAA0372 | 94828145 | 5 |
| rs1421911 | LOC389311 | 96026703 | 5 |
| rs549103 | SLAMF7 | 1.58E+08 | 1 |
| rs6814782 | PPP2R2C | 6575684 | 4 |
| rs16822974 | HLA-DRB1 | 32595192 | 6 |
| rs752637 | IRF5 | 1.28E+08 | 7 |
| rs151940 | Null | 96173533 | 5 |
| rs30168 | DNAH5 | 13772089 | 5 |
| hCV37329 | NULL | 22733807 | 4 |
| rs9285017 | FLJ25333 | 94825167 | 5 |
| rs1824794 | LOC388523 | 22881436 | 19 |
| rs2400313 | SIAT8D | 1E+08 | 5 |
| rs6842695 | KIAA0746 | 25537628 | 4 |
| rs12068654 | LOC400792 | 1.58E+08 | 1 |
| rs5017567 | FCGR2B-FCGR2C | 1.58E+08 | 1 |
| rs7096206 | MBL2 | 54201691 | 10 |
| rs4689527 | CNO | 6839049 | 4 |
| rs16972197 | TNFSF13B | 1.08E+08 | 13 |
| rs685523 | ADAMTS13 | 1.33E+08 | 9 |
| rs2746414 | C4B | 32072207 | 6 |
| rs9929801 | ITGAM | 31190973 | 16 |
| rs489286 | SLAMF7 | 1.58E+08 | 1 |
| rs518721 | SLAMF7 | 1.58E+08 | 1 |
| rs4623093 | PPP2R2C | 6633139 | 4 |
| rs17047660 | CR1 | 2.04E+08 | 1 |
| rs3741983 | PTPN11 | 1.11E+08 | 12 |
| rs13312724 | TRADD | 65749111 | 16 |
| rs7732536 | Null | 97886819 | 5 |
| rs1156556 | Null | 97887190 | 5 |
| rs2121001 | Null | 32372232 | 4 |
| rs1249550 | SLAMF6 | 1.57E+08 | 1 |
| rs1717343 | Null | 77402372 | 12 |
| rs7170637 | CYFIP1 | 20520673 | 15 |
| rs10501549 | DLG2 | 83230041 | 11 |
| rs569932 | SLAMF7 | 1.58E+08 | 1 |
| rs11805036 | SLAMF6 | 1.57E+08 | 1 |
| rs2240345 | SEC14L3 | 29181927 | 22 |
| rs27524 | ARTS-1-CAST | 96127700 | 5 |
| rs903649 | null | 41519949 | 3 |
| rs570901 | CD48/SLAMF7 | 1.58E+08 | 1 |
| rs2853690 | TERT | 1306744 | 5 |
| rs10500538 | CKLFSF1 | 65165690 | 16 |
| rs1862975 | GALNT14 | 31238083 | 2 |
| rs738546 | LOC112885 | 43753864 | 22 |
| rs1335870 | NULL | 19803776 | 13 |
| rs4698672 | LOC391636 | 13624108 | 4 |
| hCV25924532 | MRPS36P1 | 6764230 | 3 |
| rs3912216 | EVC | 5892578 | 4 |
| rs429358 | APOE | 50103781 | 19 |
| rs10489755 | FIBL-6 | 1.83E+08 | 1 |
| rs10502677 | LOC441820 | 33532401 | 18 |
| rs1872234 | KIAA1199 | 78877138 | 15 |
| rs995173 | LOC442426 | 81983513 | 9 |
| rs17841951 | HLA-DRB1 | 32657526 | 6 |
| rs1383067 | Null | 65203559 | 3 |
| rs3172604 | CNO | 6836847 | 4 |
| rs2232376 | IRX4 | 1933891 | 5 |
| rs159349 | FLJ22344 | 94278617 | 5 |
| rs766843 | GRIA3 | 1.22E+08 | X |
| rs1392581 | Null | 20808751 | 3 |
| rs7465764 | CD72 | 35604924 | 9 |
| rs915171 | EPB41L2 | 1.31E+08 | 6 |
| rs7861396 | JMJD2C | 7099706 | 9 |
| rs752211 | ZNF423/OAZ | 48086695 | 16 |
| rs6020572 | PTPN1 | 48561647 | 20 |
| rs1019117 | NOG | 52103128 | 17 |
| rs3776171 | SIAT8D | 1E+08 | 5 |
| rs1802364 | PCID1 | 32579906 | 11 |
| rs4630 | GSTT1 | 22700876 | 22 |
| rs1042206 | FCGR3A-FCGR3B | 1.58E+08 | 1 |
| rs8191449 | GSTP1 | 67108957 | 11 |
| rs4959084 | TNXA | 32084904 | 6 |
| rs396716 | FCGR3A-FCGR3B | 1.58E+08 | 1 |
| rs1528077 | NULL | 10992318 | 4 |
| rs4855518 | TAFA4 | 68941119 | 3 |
| rs4459610 | ACE | 58938452 | 17 |
| rs4689719 | SORCS2 | 7508487 | 4 |
| rs2913851 | COL23A1 | 1.78E+08 | 5 |
| rs2268277 | Runx1 | 35103919 | 21 |
| rs6474 | CYP21A2 | 32114865 | 6 |
| rs4343 | ACE | 58919763 | 17 |
| rs741441 | LOC147991 | 37590914 | 19 |
| rs10515470 | Null | 1.35E+08 | 5 |
| rs13028722 | EIF5B | 99410677 | 2 |
| rs2266637 | GSTT1 | 22701399 | 22 |
| rs1538971 | FREB | 1.58E+08 | 1 |
| rs10520774 | Null | 93556365 | 15 |
| rs670902 | MGAT1 | 1.8E+08 | 5 |
| rs1042207 | FCGR3A-FCGR3B | 1.58E+08 | 1 |
| rs1544402 | SORCS2 | 7849381 | 4 |
| rs1340831 | Null | 86950030 | 13 |
| rs4647001 | JUN | 58962439 | 1 |
| rs16899606 | MICB | 31582807 | 6 |
| rs1078887 | STK32B | 5219396 | 4 |
| rs663744 | ROCK1 | 16962488 | 18 |
| rs3761959 | FCRL3 | 1.54E+08 | 1 |
| rs7323181 | Null | 86950467 | 13 |
| rs9272711 | HLA-DQA1 | 32717290 | 6 |
| rs2976230 | ITGAE | 3577990 | 17 |
| rs2269961 | SEC14L3 | 29185384 | 22 |
| rs296067 | NULL | 1.23E+08 | 2 |
| rs596502 | RYR2 | 2.34E+08 | 1 |
| rs6501734 | RAB37 | 70247224 | 17 |
| rs1024611 | CCL2 | 29603901 | 17 |
| rs6701264 | CSMD2 | 34168985 | 1 |
| rs3738057 | SMYD3 | 2.42E+08 | 1 |
| rs6792 | LSM4 | 18279044 | 19 |
| rs4820853 | SEC14L3 | 29189164 | 22 |
| rs469736 | ARTS-1 | 96146825 | 5 |
| rs4345249 | PPP2R2C | 6545142 | 4 |
| rs7522061 | FCRH3 | 1.54E+08 | 1 |
| rs3682 | ACOX1 | 71453745 | 17 |
| R51536389 | PPAPDC1 | 1.22E+08 | 10 |
| rs231775 | CTLA4 | 2.05E+08 | 2 |
| rs10515218 | FLJ22344 | 94171011 | 5 |
| rs201638 | ACTRT1 | 1.26E+08 | X |
| rs2076529 | BTNL2 | 32471933 | 6 |
| rs32015 | Null | 66733395 | 5 |
| rs10840108 | RPL27A | 8660517 | 11 |
| rs2076530 | BTNL2 | 32471794 | 6 |
| rs763362 | CD226 | 65682777 | 18 |
| rs1790588 | CD226 | 65686164 | 18 |
| rs316208 | LNPEP | 96406729 | 5 |
| rs947894 | GSTP1 | 67109265 | 11 |
| rs2673444 | NULL | 12720687 | 4 |
| rs1800451 | MBL2 | 54201232 | 10 |
| rs1788230 | CD226 | 65684054 | 18 |
| rs1800629 | TNF | 31651010 | 6 |
| rs8054708 | CKLFSF1 | 65167378 | 16 |
| rs1846224 | ESRRG | 2.13E+08 | 1 |
| rs13857 | SIAT8D | 1E+08 | 5 |
| rs1551443 | STAT4 | 1.92E+08 | 2 |
| rs722748 | LOC341333 | 66786791 | 12 |
| rs11264799 | FCRL3 | 1.54E+08 | 1 |
| rs1912818 | LYN | 57066875 | 8 |
| rs4891786 | CD226 | 65722590 | 18 |
| rs7189121 | LOC57019 | 56029877 | 16 |
| rs896086 | CKLFSF1 | 65164689 | 16 |
| rs4703141 | Null | 97943067 | 5 |
| rs11552708 | TNFSF13 | 7403279 | 17 |
| rs3729639 | E2F4 | 65783002 | 16 |
| rs3740955 | RAG1 | 36552176 | 11 |
| rs2269920 | PPP2R2C | 6441526 | 4 |
| rs1143634 | IL1B | 1.13E+08 | 2 |
| rs4696796 | HTRA3 | 8421628 | 4 |
| rs10501554 | DLG2 | 83523505 | 11 |
| rs2854482 | AKR1C2 | 5033821 | 10 |
| rs4061077 | LYN | 57065996 | 8 |
| rs4359427 | NFATC3 | 66760248 | 16 |
| rs1829883 | Null | 98809002 | 5 |
| rs16942067 | PTPN11 | 1.11E+08 | 12 |
| rs2040309 | DLG2 | 83215652 | 11 |
| rs2838467 | TMEM1 | 44260783 | 21 |
| rs266472 | HIPK3 | 33316957 | 11 |
| rs6447872 | AFAP | 8061177 | 4 |
| rs220479 | ITGAE | 3603924 | 17 |
| rs1559059 | SIAT8D | 1E+08 | 5 |
| rs1428439 | SIAT8D | 1E+08 | 5 |
| rs5951676 | SMS | 21764485 | X |
| rs4830643 | TBL1X | 9241795 | X |
| rs762735 | TREX2 | 1.52E+08 | X |
| rs7050108 | RNF128 | 1.06E+08 | X |
| rs10521986 | DMD | 31871236 | X |
| rs647000 | AMOT | 1.12E+08 | X |
| rs10499509 | Null | 17796011 | 7 |
| rs1977364 | JARID1C | 53111169 | X |
| rs884840 | GAB3 | 1.53E+08 | X |
| rs7447673 | SIAT8D | 1.E+08 | 5 |
| rs6950894 | MLL5 | 1.04E+08 | 7 |
| rs736818 | KLHL13 | 1.16E+08 | X |
| rs576523 | LOC400792 | 1.58E+08 | 1 |
| rs3830137 | FGD1 | 54364854 | X |
| rs3913241 | STAG2 | 1.23E+08 | X |
| rs896120 | NULL | 21465794 | 4 |
| rs392610 | C4B | 32060160 | 6 |
| rs197036 | CHRDL1 | 1.1E+08 | X |
| rs997148 | CHRM3 | 2.36E+08 | 1 |
| rs699 | AGT | 2.27E+08 | 1 |
| rs4348732 | GALNT2 | 2.27E+08 | 1 |
| rs723234 | BAIAP1 | 65959378 | 3 |
| hCV513970 | CLSTN1 | 9728571 | 1 |
| rs753790 | NULL | 90878236 | 9 |
| rs17880314 | TNFRSF1B | 12183208 | 1 |
| rs9613221 | TPST2 | 25311380 | 22 |
| rs2054024 | DLG2 | 83671604 | 11 |
| rs6829169 | PPP2R2C | 6560039 | 4 |
| rs558743 | LOC391636 | 13611840 | 4 |
| rs1481196 | NULL | 12315050 | 4 |
| rs723207 | Null | 97817581 | 5 |
| rs16962017 | KIAA0256 | 47126510 | 15 |
| rs7359387 | NFAT5 | 68291166 | 16 |
| rs616634 | C4B-STK19 | 32056427 | 6 |
| rs7664714 | PPP2R2C | 6583863 | 4 |
| rs7627719 | CHL1 | 430470 | 3 |
| rs2243250 | IL4 | 1.32E+08 | 5 |
| rs1530394 | GALNT14 | 31275383 | 2 |
| rs1398103 | LOC338825 | 1.26E+08 | 12 |
| rs7546784 | FLJ11383 | 2.3E+08 | 1 |
| rs493284 | null | 13614819 | 4 |
| rs2232968 | LSM4 | 18284402 | 19 |
| rs8059662 | ELMO3 | 65791780 | 16 |
| rs486052 | DLG2 | 83813054 | 11 |
| rs4234708 | EVC2 | 5704067 | 4 |
| rs4689261 | EVC2 | 5695407 | 4 |
| rs1903346 | null | 1.37E+08 | 4 |
| rs1402043 | NULL | 12355801 | 4 |
| rs1546689 | NULL | 12360492 | 4 |
| rs870625 | NULL | 12371964 | 4 |
| rs2234978 | TNFRSF6/FAS | 90761809 | 10 |
| rs1799983 | NOS3 | 1.5E+08 | 7 |
| rs2259820 | TCF1 | 1.2E+08 | 12 |
| rs4645203 | CPZ | 8787690 | 4 |
| rs955371 | CD244/ITLN1 | 1.58E+08 | 1 |
| rs7684111 | NULL | 12100974 | 4 |
| rs2221903 | IL22 | 1.24E+08 | 4 |
| rs1943547 | Null | 26507069 | 18 |
| rs352692 | CD48/SLAMF7 | 1.57E+08 | 1 |
| rs3802894 | DLG2 | 83837193 | 11 |
| rs729302 | IRF5 | 1.28E+08 | 7 |
| rs2216832 | GALNT14 | 31260041 | 2 |
| rs4131992 | ANKRD12 | 9181420 | 18 |
| rs704409 | PRICKLE2 | 64221869 | 3 |
| rs763110 | FASLG | 1.69E+08 | 1 |
| rs1467558 | CD44 | 35186249 | 11 |
| rs3817190 | CAMKK2 | 1.2E+08 | 12 |
| rs1805015 | IL4R | 27281681 | 16 |
| rs10489639 | CD48 | 1.57E+08 | 1 |
| rs632994 | HRH2 | 1.75E+08 | 5 |
| rs16922502 | LYN | 57049990 | 8 |
| rs3753389 | CD244 | 1.58E+08 | 1 |
| rs289332 | LOC388523 | 22945590 | 19 |
| rs1161320 | Null | 99276603 | 5 |
| rs9463339 | CD2AP | 47643167 | 6 |
| rs12078645 | Roquin | 1.71E+08 | 1 |
| rs10489638 | CD48 | 1.57E+08 | 1 |
| rs1293755 | OAS2 | 1.12E+08 | 12 |
| hCV11523632 | FLJ20850 | 18556984 | 19 |
| rs4646421 | CYP1A1 | 72803245 | 15 |
| rs2070908 | TRA1 | 1.03E+08 | 12 |
| rs4944481 | DLG2 | 83710125 | 11 |
| rs1043276 | CD2AP | 47701961 | 6 |
| rs10501564 | DLG2 | 83858905 | 11 |
| rs12334430 | LYN | 57022514 | 8 |
| rs8176927 | DNASE1 | 3645381 | 16 |
| rs4689810 | SORCS2 | 7750401 | 4 |
| hCV2544039 | ACACA | 32534523 | 17 |
| rs10095917 | LYN | 56992814 | 8 |
| rs10912711 | Roquin | 1.71E+08 | 1 |
| rs1322995 | PDCD4 | 1.13E+08 | 10 |
| rs4632418 | NULL | 10880236 | 20 |
| rs6836154 | PPP2R2C | 6571139 | 4 |
| rs6882366 | Null | 95890449 | 5 |
| rs1059551 | HLA-DRB1 | 32665466 | 6 |
| rs1859330 | OAS3 | 1.12E+08 | 12 |
| rs7419145 | CD244 | 1.58E+08 | 1 |
| rs2734316 | C4B | 32072300 | 6 |
| rs11728697 | SPP1/osteopontin | 89256120 | 4 |
| rs1065411 | GSTM1 | 1.1E+08 | 1 |
| rs6689237 | SLAMF6 | 1.57E+08 | 1 |
| rs749174 | GSTP1 | 67109829 | 11 |
| rs16922441 | LYN | 57013886 | 8 |
| rs10497523 | TTN | 1.79E+08 | 2 |
| rs17011001 | GALNT14 | 31335094 | 2 |
| rs16832364 | LOC400792 | 1.58E+08 | 1 |
| rs1800351 | SGCG | 22722783 | 13 |
| rs2812152 | Null | 67200623 | 6 |
| rs2011741 | FLJ21159 | 1.57E+08 | 4 |
| rs978763 | CD48 | 1.57E+08 | 1 |
| rs6662885 | SLAMF6/CD84 | 1.57E+08 | 1 |
| rs10514350 | Null | 90939365 | 5 |
| rs869167 | CD244/ITLN1 | 1.58E+08 | 1 |
| rs224490 | HS3ST1 | 11094910 | 4 |
| rs7990 | HLA-DQA1 | 32717943 | 6 |
| rs3845627 | SLAMF6 | 1.57E+08 | 1 |
| rs1593443 | Null | 1.39E+08 | 4 |
| rs2285932 | OAS3 | 1.12E+08 | 12 |
| rs11867053 | CKLFSF1 | 65164402 | 16 |
| rs10501558 | DLG2 | 83709460 | 11 |
| rs4689148 | SORCS2 | 7755368 | 4 |
| rs757298 | EMR2 | 14704292 | 19 |
| rs1799930 | NAT2 | 18302383 | 8 |
| rs318493 | SERPINB9 | 2849790 | 6 |
| rs1145271 | DCC | 48307974 | 18 |
| rs493950 | TRIM17 | 2.25E+08 | 1 |
| rs2476601 | PTPN22 | 1.14E+08 | 1 |
| rs1503860 | SLAMF6/CD84 | 1.57E+08 | 1 |
| rs2302464 | BST1 | 15385521 | 4 |
| rs4943893 | DLG2 | 83757615 | 11 |
| rs6682654 | CD244 | 1.58E+08 | 1 |
| rs1947027 | GALNT14 | 31299608 | 2 |
| rs4808756 | IFI30 | 18149004 | 19 |
| rs10515232 | ELL2 | 95336741 | 5 |
| rs2880013 | RNF128 | 1.06E+08 | X |
| rs10027973 | PPP2R2C | 6584159 | 4 |
| rs872883 | PPP2R2C | 6582619 | 4 |
| rs1126772 | SPP1/osteopontin | 89261365 | 4 |
| rs2227973 | RAG1 | 36553889 | 11 |
| rs218867 | C6orf170 | 1.21E+08 | 6 |
| rs7594102 | GALNT14 | 31131075 | 2 |
| rs540224 | SLAMF7/LY9 | 1.58E+08 | 1 |
| rs6703547 | Roquin | 1.71E+08 | 1 |
| rs980941 | NULL | 1.23E+08 | 2 |
| rs9308914 | GALNT14 | 31175084 | 2 |
| rs6686083 | Roquin | 1.71E+08 | 1 |
| rs1801284 | ha1 | 1019738 | 19 |
| rs1527973 | NULL | 1.23E+08 | 2 |
| rs755403 | PPP2R2C | 6507714 | 4 |
| rs6049288 | SNRPB | 2399451 | 20 |
| rs4689434 | PPP2R2C | 6523873 | 4 |
| rs4952038 | GALNT14 | 31184643 | 2 |
| rs3803800 | TNFSF13 | 7403693 | 17 |
| rs2904880 | CD19 | 28851897 | 16 |
| rs7530661 | Roquin | 1.71E+08 | 1 |
| rs887565 | NULL | 14026067 | 4 |
| rs2240188 | OAS3 | 1.12E+08 | 12 |
| rs966240 | NULL | 11318803 | 4 |
| rs746158 | ZNF423/OAZ | 48151027 | 16 |
| rs7192 | HLA-DRA | 32519624 | 6 |
| rs12928810 | ITGAM | 31219216 | 16 |
| rs909253 | LTA | 31648292 | 6 |
| rs1041981 | LTA | 31648763 | 6 |
| rs2076523 | BTNL2 | 32478813 | 6 |
| rs223825 | CCL22 | 55957472 | 16 |
| rs2008535 | GALNT14 | 31272331 | 2 |
| rs907715 | IL22 | 1.24E+08 | 4 |
| rs7499077 | ITGAM | 31225006 | 16 |
| rs223818 | CCL22 | 55952259 | 16 |
| rs241447 | Tap2 | 32904729 | 6 |
| rs10759 | RGS4 | 1.6E+08 | 1 |
| rs241448 | TAP2 | 32904663 | 6 |
| rs1367534 | DLG2 | 83894200 | 11 |
| rs6543606 | GALNT14 | 31266159 | 2 |
| rs910925 | GEMIN4 | 596297 | 17 |
| rs956747 | GALNT14 | 31286384 | 2 |
| rs1562023 | GALNT14 | 31282483 | 2 |
| rs1400657 | STAT1 | 1.92E+08 | 2 |
| rs10494382 | RGS5 | 1.6E+08 | 1 |
| rs2080338 | Null | 96629329 | 7 |
| rs953121 | DCC | 49192822 | 18 |
| rs6543607 | GALNT14 | 31277894 | 2 |
| rs12720356 | TYK2 | 10330975 | 19 |
| rs7813271 | LYN | 57025705 | 8 |
| rs6840362 | SPP1/osteopontin | 89257099 | 4 |
| rs2295614 | SLAMF1 | 1.57E+08 | 1 |
| rs2304256 | TYK2 | 10336652 | 19 |
| rs11727636 | MAN2B2 | 6675343 | 4 |
| rs4952026 | GALNT14 | 31152777 | 2 |
| rs4926508 | KIAA1720 | 2.45E+08 | 1 |
| rs2857713 | LTA | 31648535 | 6 |
| rs3796504 | SLAMF1 | 1.57E+08 | 1 |
| rs3764809 | MAN2B2 | 6698355 | 4 |
| rs1384753 | DLG2 | 83333212 | 11 |
| rs272750 | Null | 33381837 | 5 |
| rs2267575 | CD22 | 40517045 | 19 |
| rs2272229 | ANK2 | 1.15E+08 | 4 |
| rs9308917 | GALNT14 | 31305900 | 2 |
| rs7140646 | STRN3 | 30454974 | 14 |
| rs1799969 | ICAM1 | 10255792 | 19 |
| rs10494344 | SLAMF6 | 1.57E+08 | 1 |
| rs2161258 | DKFZp313G1735 | 94929209 | 5 |
| rs1187468 | Null | 4118697 | 5 |
| rs2044246 | GALNT14 | 31307667 | 2 |
| rs11265416 | SLAMF6 | 1.57E+08 | 1 |
| rs4031871 | Null | 96773239 | 5 |
| rs1050152 | SLC22A4 | 1.32E+08 | 5 |
| rs10501561 | DLG2 | 83785665 | 11 |
| rs4075082 | PFKFB4 | 48530086 | 3 |
| rs16849611 | ZNFN1A2 | 2.14E+08 | 2 |
| rs7693333 | MGC16169 | 1.07E+08 | 4 |
| rs12964890 | CD226 | 65707665 | 18 |
| rs9930690 | ITGAM | 31249753 | 16 |
| rs12025852 | SLAMF6 | 1.57E+08 | 1 |
| rs10514339 | MASS1 | 90183692 | 5 |
| rs2667979 | LYN | 57060355 | 8 |
| rs743572 | cyp17a1 | 1.05E+08 | 10 |
| rs2288101 | GALNT14 | 31046835 | 2 |
| rs8058614 | ELMO3 | 65791198 | 16 |
| rs1332612 | PAPD1 | 30474807 | 10 |
| rs13312727 | TRADD | 65745944 | 16 |
| rs3741981 | OAS1 | 1.12E+08 | 12 |
| rs1945831 | DLG2 | 83463934 | 11 |
| rs10516799 | SPP1/osteopontin | 89260372 | 4 |
| rs2258218 | C4B | 32071538 | 6 |
| rs1449613 | Null | 61873325 | 4 |
| rs1790932 | CD226 | 65684380 | 18 |
| rs2304974 | PSMB6 | 4647951 | 17 |
| rs4592896 | GALNT14 | 31311639 | 2 |
| rs997669 | CCNE1 | 34996323 | 19 |
| rs763361 | CD226 | 65682622 | 18 |
| rs7111775 | DLG2 | 84306661 | 11 |
| rs1209412 | ARHGEF5 | 1.43E+08 | 7 |
| rs3852121 | SORCS2 | 7510595 | 4 |
| rs7678146 | GPM6A | 1.77E+08 | 4 |
| rs922388 | Null | 18257970 | 4 |
| rs574610 | LY9 | 1.58E+08 | 1 |
| rs7572482 | STAT4 | 1.92E+08 | 2 |
| rs17812659 | LYN | 57052416 | 8 |
| rs1957020 | AKAP6 | 31989126 | 14 |
| rs867496 | GALNT14 | 31142484 | 2 |
| rs727088 | CD226 | 65681419 | 18 |
| rs1075760 | SORCS2 | 7618466 | 4 |
| rs6811536 | SPP1/osteopontin | 89259584 | 4 |
| rs1980606 | CD48 | 1.57E+08 | 1 |
| rs1131877 | TRAF3 | 1.02E+08 | 14 |
| rs571841 | LY9 | 1.58E+08 | 1 |
| rs2719244 | LYN | 57046135 | 8 |
| rs6471 | CYP21A1P | 32083129 | 6 |
| rs7743647 | C4B-STK19 | 32056488 | 6 |
| rs3774820 | STK32B | 5579130 | 4 |
| rs1985413 | PPP2R2C | 6568034 | 4 |
| rs2822433 | LOC375108 | 14439974 | 21 |
| rs351453 | EAT2 | 1.59E+08 | 1 |
| rs4689455 | PPP2R2C | 6584396 | 4 |
| hCV25964951 | DHX37 | 1.24E+08 | 12 |
| rs2822432 | LOC375108 | 14438819 | 21 |
| rs4404624 | PPP2R2C | 6578611 | 4 |
| rs194302 | NKAP | 1.19E+08 | X |
| rs485199 | DLG2 | 83012150 | 11 |
| rs6813956 | PPP2R2C | 6561003 | 4 |
| rs7377023 | PPP2R2C | 6625913 | 4 |
| rs10488100 | Null | 51586655 | 7 |
| rs3088063 | CD22 | 40529916 | 19 |
| rs3744165 | SLC9A3R1 | 78383731 | 17 |
| rs1800471 | TGFB1 | 46550716 | 19 |
| rs4094864 | CD226 | 65696478 | 18 |
| rs11151544 | CD226 | 65697070 | 18 |
| rs731196 | NULL | 32034765 | 4 |
| rs4443273 | NULL | 14484953 | 4 |
| rs4254932 | CAST | 96000943 | 5 |
| rs2933572 | MSX1 | 4917764 | 4 |
| rs743351 | PCBP3 | 46110723 | 21 |
| rs1788112 | CD226 | 65716878 | 18 |
| rs4696765 | ABLIM2 | 8243859 | 4 |
| rs4505896 | PPP2R2C | 6617188 | 4 |
| rs2055979 | IL22 | 1.24E+08 | 4 |
| rs6583288 | TFRC | 1.97E+08 | 3 |
| rs164288 | SLAMF1 | 1.57E+08 | 1 |

6. Oligonucleotide Synthesis

Oligonucleotide synthesis is well known to those of skill in the art. Various mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which is incorporated herein by reference in its entirety.

Basically, chemical synthesis can be achieved by the diester method, the triester method polynucleotides phosphorylase method and by solid-phase chemistry. These methods are discussed in further detail below.

Diester Method.

The diester method was the first to be developed to a usable state, primarily by Khorana and co-workers (Khorana, 1979). The basic step is the joining of two suitably protected deoxynucleotides to form a dideoxynucleotide containing a phosphodiester bond. The diester method is well established and has been used to synthesize DNA molecules (Khorana, 1979).

Triester Method.

The main difference between the diester and triester methods is the presence in the latter of an extra protecting group on the phosphate atoms of the reactants and products (Itakura et al., 1975). The phosphate protecting group is usually a chlorophenyl group, which renders the nucleotides and polynucleotide intermediates soluble in organic solvents. Therefore, purifications are done in chloroform solutions. Other improvements in the method include (i) the block coupling of trimers and larger oligomers, (ii) the extensive use of high-performance liquid chromatography for the purification of both intermediate and final products, and (iii) solid-phase synthesis.

Polynucleotide Phosphorylase Method.

This is an enzymatic method of DNA synthesis that can be used to synthesize many useful oligodeoxynucleotides (Gillam et al., 1978). Under controlled conditions, polynucleotide phosphorylase adds predominantly a single nucleotide to a short oligodeoxynucleotide. Chromatographic purification allows the desired single adduct to be obtained. At least a trimer is required to initiate the method of adding one base at a time, a primer that must be obtained by some other method. The polynucleotide phosphorylase method works and has the advantage that the procedures involved are familiar to most biochemists.

Solid-Phase Methods.

The technology developed for the solid-phase synthesis of polypeptides has been applied after an, it has been possible to attach the initial nucleotide to solid support material has been attached by proceeding with the stepwise addition of nucleotides. All mixing and washing steps are simplified, and the procedure becomes amenable to automation. These syntheses are now routinely carried out using automatic DNA synthesizers.

Phosphoramidite chemistry (Beaucage, 1993) has become by far the most widely used coupling chemistry for the synthesis of oligonucleotides. As is well known to those skilled in the art, phosphoramidite synthesis of oligonucleotides involves activation of nucleoside phosphoramidite monomer precursors by reaction with an activating agent to form activated intermediates, followed by sequential addition of the activated intermediates to the growing oligonucleotide chain (generally anchored at one end to a suitable solid support) to form the oligonucleotide product.

7. Separation of Nucleic Acids

In certain embodiments, nucleic acid products are separated by agarose, agarose-acrylamide or polyacrylamide gel electrophoresis using standard methods (Sambrook et al., 1989). Separated products may be cut out and eluted from the gel for further manipulation. Using low melting point agarose gels, the skilled artisan my remove the separated band by heating the gel, followed by extraction of the nucleic acid.

Separation of nucleic acids may also be effected by chromatographic techniques known in the art. There are many kinds of chromatography that may be used in the practice of the present invention, including capillary adsorption, partition, ion-exchange, hydroxylapatite, molecular sieve, reverse-phase, column, paper, thin-layer, and gas chromatography as well as HPLC.

A number of the above separation platforms can be coupled to achieve separations based on two different properties. For example, some of the primers can be coupled with a moiety that allows affinity capture, and some primers remain unmodified. Modifications can include a sugar (for binding to a lectin column), a hydrophobic group (for binding to a reverse-phase column), biotin (for binding to a streptavidin column), or an antigen (for binding to an antibody column). Samples are run through an affinity chromatography column. The flow-through fraction is collected, and the bound fraction eluted (by chemical cleavage, salt elution, etc.). Each sample is then further fractionated based on a property, such as mass, to identify individual components.

### II. Autoimmune Disease

A. Systemic Lupus Erythematosus

1. Definition and Symptoms

Systemic lupus erythematosus (SLE) is an autoimmune chronic inflammatory disease that most commonly affects the skin, joints, kidneys, heart, lungs, blood vessels, and brain. The most common symptoms include fatigue, muscle aches, low-grade fever, skin rashes, and kidney problems that are sometimes severe enough to require dialysis or transplant. Symptoms may also include a characteristic facial rash (“butterfly rash”), photosensitivity, and poor circulation to the extremities with cold exposure, known as Raynaud's phenomenon. Rheumatoid arthritis is another chronic autoimmune disease, and most people with SLE will develop arthritis during the course of their illness with similar symptoms to rheumatoid arthritis. Because SLE can affect the walls of the blood vessels, young women with SLE are at significantly higher risk for heart attacks from coronary artery disease. For many patients, alopecia occurs as SLE worsens.

Women who become pregnant with SLE are considered “high risk.” These women have an increased risk of miscarriages, and the incidence of flares can increase with pregnancy. Antibodies from SLE can be transferred to the fetus, resulting in “neonatal lupus.” Symptoms of neonatal lupus include anemia and skin rash, with congenital heart block being less common. Unlike SLE, neonatal lupus resolves after six months as the newborn metabolizes the mother's antibodies.

2. Diagnosis

Because the symptoms of SLE can vary widely, accurate diagnosis is difficult. A diagnosis of SLE is suggested for a patient who meets four or more of the eleven criteria established by the American Rheumatism Association, but there is currently no single test that establishes the diagnosis of SLE. However, these criteria are not definitive. The criteria are based on the symptoms of SLE, but also include the presence of anti-DNA, antinuclear (ANA), or anti-Sm antibodies, a false positive test for syophilis, anticardiolipin antibodies, lupus anticoagulant, or positive LE prep test. Some patients are diagnosed with SLE who manifest fewer than four criteria, while other such patients remain undiagnosed.

Most people with SLE test positive for ANA. Even so, the test is not definitive, as a number of conditions can cause a positive ANA test. Other antibody tests that can aid in a diagnosis of SLE or other autoimmune conditions include anti-RNP, anti-Ro (SSA), and anti-La (SSB).

3. Treatment

There is currently no cure for SLE, and the illness remains characterized by alternating periods of illness, or flares, and periods of wellness, or remission. The current goal of treatment is to relieve the symptoms of SLE, and to protect the organ systems affected by decreasing the level of autoimmune activity. More and better quality rest is prescribed for fatigue, along with exercise to maintain joint strength and range of motion. DHEA (dehydroepiandrosterone) can reduce fatigue and thinking problems associated with SLE. Physicians also commonly prescribe Nonsteroidal antiinflammatory drugs (NSAIDs) for pain and inflammation, although this can cause stomach pain and even ulcers in some patients.

Hydroxychloroquine, an anti-malarial medication, can be effective in treating fatigue related to SLE as well as skin and joint problems. Hydroxychloroquine also decreases the frequency of excessive blood clotting in some SLE patients. Corticosteroids are needed for more serious cases, although the serious side effects, such as weight gain, loss of bone mass, infection, and diabetes limits the length of time and dosages at which they can be prescribed. Immunosuppressants, or cytotoxic drugs, are used to treat severe cases of SLE, but again serious side effects such as increased risk of infection from decreased blood cell counts are common.

Possible future therapies include stem cell transplants to replace damaged immune cells and radical treatments that would temporarily kill all immune system cells. Other future treatments may include “biologic agents” such as the genetically engineered antibody rituximab (anti-CD20) that block parts of the immune system, such as B cells. Recently, two groups of researchers found that even partial restoration of function of an inhibitory Fc receptor prevented the development of SLE in several strains of mice that were genetically prone to the disease. Reviewed in Kuehn, Lupus (2005).

4. Who SLE Affects

SLE is much more common among women than men, with women comprising approximately 90% of all SLE patients. It is also three times more common in African American women than in women of European descent, although the incidence is also higher among women of Japanese and Chinese ancestry.

Because widely varying symptoms of SLE make accurate diagnosis difficult, the exact number of people who suffer from SLE is unknown. The Lupus Foundation of America, however, estimates that approximately 1,500,000 Americans have some form of lupus. The prevalence of SLE is estimated to be about 40 per 100,000.

B. Other Autoimmune Diseases

1. Rheumatoid Arthritis

The exact etiology of RA remains unknown, but the first signs of joint disease appear in the synovial lining layer, with proliferation of synovial fibroblasts and their attachment to the articular surface at the joint margin (Lipsky, 1998). Subsequently, macrophages, T cells and other inflammatory cells are recruited into the joint, where they produce a number of mediators, including the cytokines interleukin-1 (IL-1), which contributes to the chronic sequelae leading to bone and cartilage destruction, and tumour necrosis factor (TNF-α), which plays a role in inflammation (Dinarello, 1998; Arend & Dayer, 1995; van den Berg, 2001). The concentration of IL-1 in plasma is significantly higher in patients with RA than in healthy individuals and, notably, plasma IL-1 levels correlate with RA disease activity (Eastgate et al., 1988). Moreover, synovial fluid levels of IL-1 are correlated with various radiographic and histologic features of RA (Kahle et al., 1992; Rooney et al., 1990).

In normal joints, the effects of these and other proinflammatory cytokines are balanced by a variety of anti-inflammatory cytokines and regulatory factors (Burger & Dayer, 1995). The significance of this cytokine balance is illustrated in juvenile RA patients, who have cyclical increases in fever throughout the day (Prieur et al., 1987). After each peak in fever, a factor that blocks the effects of IL-1 is found in serum and urine. This factor has been isolated, cloned and identified as IL-1 receptor antagonist (IL-1ra), a member of the IL-1 gene family (Hannum et al., 1990). IL-1ra, as its name indicates, is a natural receptor antagonist that competes with IL-1 binding to type I IL-1 receptors and, as a result, blocks the effects of IL-1 (Arend et al., 1998). A 10- to 100-fold excess of IL-1ra may be needed to block IL-1 effectively; however, synovial cells isolated from patients with RA do not appear to produce enough IL-1ra to counteract the effects of IL-1 (Firestein et al., 1994; Fujikawa et al., 1995).

2. Sjögren's Syndrome

Primary Sjögren's syndrome (SS) is a chronic, slowly progressive, systemic autoimmune disease, which affects predominantly middle-aged women (female-to-male ratio 9:1), although it can be seen in all ages including childhood (Jonsson et al., 2002). It is characterized by lymphocytic infiltration and destruction of the exocrine glands, which are infiltrated by mononuclear cells including CD4+, CD8+ lymphocytes and B-cells (Jonsson et al., 2002). In addition, extraglandular (systemic) manifestations are seen in one-third of patients (Jonsson et al., 2001).

The glandular lymphocytic infiltration is a progressive feature (Jonsson et al., 1993), which, when extensive, may replace large portions of the organs. Interestingly, the glandular infiltrates in some patients closely resemble ectopic lymphoid microstructures in the salivary glands (denoted as ectopic germinal centers) (Salomonsson et al., 2002; Xanthou & Polihronis, 2001). In SS, ectopic GCs are defined as T and B cell aggregates of proliferating cells with a network of follicular dendritic cells and activated endothelial cells. These GC-like structures formed within the target tissue also portray functional properties with production of autoantibodies (anti-Ro/SSA and anti-La/SSB) (Salomonsson &, Jonsson, 2003).

In other systemic autoimmune diseases, such as RA, factors critical for ectopic GCs have been identified. Rheumatoid synovial tissues with GCs were shown to produce chemokines CXCL13, CCL21 and lymphotoxin (LT)-β (detected on follicular center and mantle zone B cells). Multivariate regression analysis of these analytes identified CXCL13 and LT-β as the solitary cytokines predicting GCs in rheumatoid synovitis (Weyand & Goronzy, 2003). Recently CXCL13 and CXCR5 in salivary glands has been shown to play an essential role in the inflammatory process by recruiting B and T cells, therefore contributing to lymphoid neogenesis and ectopic GC formation in SS (Salomonsson et al., 2002.)

3. Autoimmune Diseases

The following is a list of autoimmune diseases which also may be subject to analysis using the SNPs listed in Tables X and Z: juvenile onset diabetes mellitus, Wegener's granulomatosis, inflammatory bowel disease, polymyositis, dermatomyositis, multiple endocrine failure, Schmidt's syndrome, autoimmune uveitis, Addison's disease, adrenalitis, Graves' disease, thyroiditis, Hashimoto's thyroiditis, autoimmune thyroid disease, pernicious anemia, gastric atrophy, chronic hepatitis, lupoid hepatitis, atherosclerosis, presenile dementia, demyelinating diseases, multiple sclerosis, subacute cutaneous lupus erythematosus, hypoparathyroidism, Dressler's syndrome, myasthenia gravis, autoimmune thrombocytopenia, idiopathic thrombocytopenic purpura, hemolytic anemia, pemphigus vulgaris, pemphigus, dermatitis herpetiformis, alopecia arcata, pemphigoid, scleroderma, progressive systemic sclerosis, CREST syndrome (calcinosis, Raynaud's phenomenon, esophageal dysmotility, sclerodactyly, and telangiectasia), adult onset diabetes mellitus (Type II diabetes), male and female autoimmune infertility, ankylosing spondolytis, ulcerative colitis, Crohn's disease, mixed connective tissue disease, polyarteritis nedosa, systemic necrotizing vasculitis, juvenile onset rheumatoid arthritis, glomerulonephritis, atopic dermatitis, atopic rhinitis, Goodpasture's syndrome, Chagas' disease, sarcoidosis, rheumatic fever, asthma, recurrent abortion, anti-phospholipid syndrome, farmer's lung, erythema multiforme, post cardiotomy syndrome, Cushing's syndrome, autoimmune chronic active hepatitis, bird-fancier's lung, allergic disease, allergic encephalomyelitis, toxic epidermal necrolysis, alopecia, Alport's syndrome, alveolitis, allergic alveolitis, fibrosing alveolitis, interstitial lung disease, erythema nodosum, pyoderma gangrenosum, transfusion reaction, leprosy, malaria, leishmaniasis, trypanosomiasis, Takayasu's arteritis, polymyalgia rheumatica, temporal arteritis, schistosomiasis, giant cell arteritis, ascariasis, aspergillosis, Sampter's syndrome, eczema, lymphomatoid granulomatosis, Behcet's disease, Caplan's syndrome, Kawasaki's disease, dengue, encephalomyelitis, endocarditis, endomyocardial fibrosis, endophthalmitis, erythema elevatum et diutinum, psoriasis, erythroblastosis fetalis, eosinophilic faciitis, Shulman's syndrome, Felty's syndrome, filariasis, cyclitis, chronic cyclitis, heterochronic cyclitis, Fuch's cyclitis, IgA nephropathy, Henoch-Schonlein purpura, glomerulonephritis, graft versus host disease, transplantation rejection, human immunodeficiency virus infection, echovirus infection, cardiomyopathy, Alzheimer's disease, parvovirus infection, rubella virus infection, post vaccination syndromes, congenital rubella infection, Hodgkin's and Non-Hodgkin's lymphoma, renal cell carcinoma, multiple myeloma, Eaton-Lambert syndrome, relapsing polychondritis, malignant melanoma, cryoglobulinemia, Waldenstrom's macroglobulemia, Epstein-Barr virus infection, mumps, Evan's syndrome, and autoimmune gonadal failure.

TABLE Z

| SNPNAME | GENE | | | | |
|---|---|---|---|---|---|
| Published/rs933564 | 16 ZNF423 | 48215132 | 0 | 0 | 0 |
| Published/rs2080353 | 16 ZNF423 | 48216025 | 0 | 0 | 0 |
| Published/rs1345431 | 16 ZNF423 | 48184356 | 0 | 0 | 0 |
| Published/rs2228570 | 12 VDR | 46559162 | 0 | 0 | 0 |
| Published/rs731236 | 12 VDR | 46525024 | 0 | 0 | 0 |
| Published/rs2238136 | 12 VDR | 46563980 | 0 | 0 | 0 |
| Published/sl544410 | 12 VDR | 46526102 | 0 | 0 | 0 |
| Published/rs2304256 | 19 TYK2 | 10336652 | 0 | 0 | 0 |
| Published/rs12720351 | 19 TYK2 | 10330975 | 0 | 0 | 0 |
| Published/rs280497 | 19 TYK2 | 10325687 | 0 | 0 | 0 |
| Published/rs915956 | 11 TRIM21 | 4366473 | 0 | 0 | 0 |
| Published/rs7947461 | 11 TRIM21 | 4368829 | 0 | 0 | 0 |
| Published/rs2280714 | 7 TNPO3 | 1.28E+08 | 0 | 0 | 0 |
| Published/rs3803800 | 17 TNFSF13 | 7403693 | 0 | 0 | 0 |
| Published/rs1155270 | 17 TNFSF13 | 7403279 | 0 | 0 | 0 |
| Published/rs361525 | 6 TNF | 31651080 | 0 | 0 | 0 |
| Published/rs1800629 | 6 TNF | 31651010 | 0 | 0 | 0 |
| Published/rs1982073 | 19 TGFB1 | 46550761 | 0 | 0 | 0 |
| Published/rs1800820 | 19 TGFB1 | 46552615 | 0 | 0 | 0 |
| Published/rs1800472 | 19 TGFB1 | 46539700 | 0 | 0 | 0 |
| Published/rs1800471 | 19 TGFB1 | 46550716 | 0 | 0 | 0 |
| Published/rs1800465 | 19 TGFB1 | 46552136 | 0 | 0 | 0 |
| Published/rs1800468 | 19 TGFB1 | 46552427 | 0 | 0 | 0 |
| Published/rs1344734 | 19 TGFB1 | 46550650 | 0 | 0 | 0 |
| Published/rs1155122 | 19 TGFB1 | 46529924 | 0 | 0 | 0 |
| Published/rs1146635 | 19 TGFB1 | 46529915 | 0 | 0 | 0 |
| Published/rs4148873 | 6 Tap2 | 32908390 | 0 | 0 | 0 |
| Published/rs241447 | 6 Tap2 | 32904729 | 0 | 0 | 0 |
| Published/rs1800573 | 6 Tap2 | 32905762 | 0 | 0 | 0 |
| Published/rs4148880 | 6 Tap1 | 32926752 | 0 | 0 | 0 |
| Published/rs1800453 | 6 Tap1 | 32922953 | 0 | 0 | 0 |
| Published/rs1914408 | 2 STAT1 | 1.92E+08 | 0 | 0 | 0 |
| Published/rs1400657 | 2 STAT1 | 1.92E+08 | 0 | 0 | 0 |
| Published/rs3841116 | 4 SPPlostec | 89253887 | 0 | 0 | 0 |
| Published/rs9138 | 4 SPP1 | 89362737 | 0 | 0 | 0 |
| Published/rs1126616 | 4 SPP1 | 89261032 | 0 | 0 | 0 |
| Published/rs3744165 | 17 SLC9A3R1 | 78383731 | 0 | 0 | 0 |
| PublishedIrs1050152 | 5 SLC22A4 | 1.32E+08 | 0 | 0 | 0 |
| Published/rs6878654 | 5 SLC22A4 | 1.32E+08 | 0 | 0 | 0 |
| Published/rs3792876 | 5 SLC22A4 | 1.32E+08 | 0 | 0 | 0 |
| Published/rs2073838 | 5 SLC22A4 | 1.32E+08 | 0 | 0 | 0 |
| Published/rs1799889 | 7 SERPlNEl | 100363145 | 0 | 0 | 0 |
| Published/rs1799768 | 7 SERPlNEl | 1E+08 | 0 | 0 | 0 |
| Published/rs5361 | 1 SELE | 1.66E+08 | 0 | 0 | 0 |
| Published/rs3741240 | 11 SCGBlAl | 61943118 | 0 | 0 | 0 |
| Published/rs1800593 | 11 SCGBlAl | 61943123 | 0 | 0 | 0 |
| Published/rs1154944 | 11 SCGBlAl | 61943118 | 0 | 0 | 0 |
| Published/rs2268277 | 21 Runx1 | 35103919 | 0 | 0 | 0 |
| Published/rs2019154 | 17 RAPTOR | 76268939 | 0 | 0 | 0 |
| Published/rs2476601 | 1 PTPN22 | 1.14E+08 | 0 | 0 | 0 |
| Published/rs1341239 | 6 PRL | 22412183 | 0 | 0 | 0 |
| Published/rs6749527 | 2 PDCD1 | | 0 | 0 | 0 |
| Published/rs6705653 | 2 PDCD1 | | 0 | 0 | 0 |
| Published/rs5839828 | 2 PDCD1 | | 0 | 0 | 0 |
| Published/rs2227981 | 2 PDCD1 | | 0 | 0 | 0 |
| Published/rs1156882 | 2 PDCD1 | | 0 | 0 | 0 |
| Published/rs1020452 | 2 PDCD1 | | 0 | 0 | 0 |
| Published/rs874881 | 1 PAD14 | 17405805 | 0 | 0 | 0 |
| Published/rs1120336 | 1 PAD14 | 17402922 | 0 | 0 | 0 |
| Published/rs1120336 | 1 PAD14 | 17402840 | 0 | 0 | 0 |
| Published/rs2240339 | 1 PAD14 | 17419414 | 0 | 0 | 0 |
| Published/rs1748033 | 1 PAD14 | 17407968 | 0 | 0 | 0 |
| Published/rs6196 | 5 NR3C1 | 1.43E+08 | 0 | 0 | 0 |
| Published/rs1799983 | 7 NOS3 | 1.5E+08 | 0 | 0 | 0 |
| Published/rs2070744 | 7 NOS3 | 1.5E+08 | 0 | 0 | 0 |
| Published/rs1095229 | 7 NOS3 | 1.5E+08 | 0 | 0 | 0 |
| Published/rs1799931 | 8 NAT2 | 18302650 | 0 | 0 | 0 |
| Published/rs1799930 | 8 NAT2 | 18302383 | 0 | 0 | 0 |
| Published/rs1208 | 8 NAT2 | 18302596 | 0 | 0 | 0 |
| Published/rs1799929 | 8 NAT2 | 18302274 | 0 | 0 | 0 |
| Published/rs1562444 | 11 MTNR1B | 92355497 | 0 | 0 | 0 |
| Published/rs9279200 | 6 MICA | 31488142 | 0 | 0 | 0 |
| Published/rs5030737 | 10 MBL2 | 54201248 | 0 | 0 | 0 |
| Published/rs1800451 | 10 MBL2 | 54201232 | 0 | 0 | 0 |
| Published/rs1800450 | 10 MBL2 | 54201241 | 0 | 0 | 0 |
| Published/rs930508 | 10 MBL2 | 54198304 | 0 | 0 | 0 |
| Published/rs7095891 | 10 MBL2 | 54201467 | 0 | 0 | 0 |
| Published/rs4647963 | 10 MBL2 | 54201445 | 0 | 0 | 0 |
| Published/rs909253 | 6 LTA | 31648292 | 0 | 0 | 0 |
| Published/rs310229 | 1 JAK1 | 65033409 | 0 | 0 | 0 |
| Published/rs310227 | 1 JAK1 | 65035163 | 0 | 0 | 0 |
| Published/rs5918 | 17 ITGB3 | 42715729 | 0 | 0 | 0 |
| Published/rs752637 | 7 IRF5IRF5 | 1.28E+08 | 0 | 0 | 0 |
| Published/rs2004640 | 7 IRF5IRF5 | 1.28E+08 | 0 | 0 | 0 |
| Published/rs729302 | 7 IRF5 | 1.28E+08 | 0 | 0 | 0 |
| Published/rs3807306 | 7 IRF5 | 1.28E+08 | 0 | 0 | 0 |
| Published/rs1874328 | 7 IRF5 | 1.28E+08 | 0 | 0 | 0 |
| Published/rs1800795 | 7 IL6 | 22539885 | 0 | 0 | 0 |
| Published/rs2234898 | 16 IL4R | 27281416 | 0 | 0 | 0 |
| Published/rs6413500 | 16 IL4R | 27281334 | 0 | 0 | 0 |
| Published/rs4787952 | 16 IL4R | 27265319 | 0 | 0 | 0 |
| Published/rs3024678 | 16 IL4R | 27282197 | 0 | 0 | 0 |
| Published/rs3024677 | 16 IL4R | 27281909 | 0 | 0 | 0 |
| Published/rs1805016 | 16 IL4R | 27282428 | 0 | 0 | 0 |
| Published/rs1805015 | 16 IL4R | 27281681 | 0 | 0 | 0 |
| Published/rs1805014 | 16 IL4R | 27282530 | 0 | 0 | 0 |
| Published/rs1805013 | 16 IL4R | 27281481 | 0 | 0 | 0 |
| Published/rs1805012 | 16 IL4R | 27281465 | 0 | 0 | 0 |
| Published/rs1805011 | 16 IL4R | 27281373 | 0 | 0 | 0 |
| Published/rs1805010 | 16 IL4R | 27263704 | 0 | 0 | 0 |
| Published/rs1801275 | 16 IL4R | 27281901 | 0 | 0 | 0 |
| Published/rs3024679 | 16 IL4R | 27282571 | 0 | 0 | 0 |
| Published/rs3024638 | 16 IL4R | 27274727 | 0 | 0 | 0 |
| Published/rs3024571 | 16 IL4R | 27265428 | 0 | 0 | 0 |
| Published/rs2234923 | 16 IL4R | 27281470 | 0 | 0 | 0 |
| Published/rs2234900 | 16 IL4R | 27281473 | 0 | 0 | 0 |
| Published/rs2234899 | 16 IL4R | 27281467 | 0 | 0 | 0 |
| Published/rs2234897 | 16 IL4R | 27281113 | 0 | 0 | 0 |
| Published/rs2234896 | 16 IL4R | 27277789 | 0 | 0 | 0 |
| Published/rs2107356 | 16 IL4R | 27230905 | 0 | 0 | 0 |
| Published/rs2057768 | 16 IL4R | 27229596 | 0 | 0 | 0 |
| Published/rs2243281 | 5 IL4 | 1.32E+08 | 0 | 0 | 0 |
| Published/rs2243280 | 5 IL4 | 1.32E+08 | 0 | 0 | 0 |
| Published/rs2243250 | 5 IL4 | 1.32E+08 | 0 | 0 | 0 |
| Published/rs4252023 | 2 IL1RN | 1.14E+08 | 0 | 0 | 0 |
| Published/rs419598 | 2 IL1RN | 1.14E+08 | 0 | 0 | 0 |
| Published/rs315952 | 2 IL1RN | 1.14E+08 | 0 | 0 | 0 |
| Published/rs2232355 | 2 IL1RN | 1.14E+08 | 0 | 0 | 0 |
| Published/rs16944 | 2 IL1B | 1.I3E+08 | 0 | 0 | 0 |
| Published/rs1143634 | 2 IL1B | 1.I3E+08 | 0 | 0 | 0 |
| Published/rs1143633 | 2 IL1B | 1.I3E+08 | 0 | 0 | 0 |
| Published/rs1143627 | 2 IL1B | 1.I3E+08 | 0 | 0 | 0 |
| Published/rs17561 | 2 IL1A | 1.I3E+08 | 0 | 0 | 0 |
| Published/rs1899587 | 18 IL1A | 68020576 | 0 | 0 | 0 |
| Published/rs7349077 | 1 IL10 | 2.03E+08 | 0 | 0 | 0 |
| Published/rs6703630 | 1 IL10 | 2.03E+08 | 0 | 0 | 0 |
| Published/rs6693899 | 1 IL10 | 2.03E+08 | 0 | 0 | 0 |
| Published/rs1800896 | 1 IL10 | 2.03E+08 | 0 | 0 | 0 |
| Published/rs1800892 | 1 IL10 | 2.03E+08 | 0 | 0 | 0 |
| Published/rs1800890 | 1 IL10 | 2.03E+08 | 0 | 0 | 0 |
| Published/rs1800872 | 1 IL10 | 2.03E+08 | 0 | 0 | 0 |
| Published/rs1800871 | 1 IL10 | 2.03E+08 | 0 | 0 | 0 |
| Published/rs9808753 | 21 IFNGR2 | 33709182 | 0 | 0 | 0 |
| Published/rs1157593 | 6 IFNGR1 | 1.38E+08 | 0 | 0 | 0 |
| Published/rs2073362 | 21 IFNAR2 | 33542671 | 0 | 0 | 0 |
| Published/rs2257167 | 21 IFNAR1 | 33637569 | 0 | 0 | 0 |
| Published/rs5498 | 19 ICAM1 | 10256683 | 0 | 0 | 0 |
| Published/rs1799969 | 19 1CAM1 | 10255792 | 0 | 0 | 0 |
| Published/rs1061581 | 6 HSPA1B | 31904759 | 0 | 0 | 0 |
| Published/rs2308776 | 6 HLA-DRB1 | 32638177 | 0 | 0 | 0 |
| Published/rs2308775 | 6 HLA-DRB1 | 32638178 | 0 | 0 | 0 |
| Published/rs2308774 | 6 HLA-DRB1 | 32638183 | 0 | 0 | 0 |
| Published/rs2308773 | 6 HLA-DRB1 | 32638185 | 0 | 0 | 0 |
| Published/rs2308771 | 6 HLA-DRB1 | 32638228 | 0 | 0 | 0 |
| Published/rs2308769 | 6 HLA-DRB1 | 32638251 | 0 | 0 | 0 |
| Published/rs2308768 | 6 HLA-DRB1 | 32638257 | 0 | 0 | 0 |
| Published/rs2308766 | 6 HLA-DRB1 | 32638277 | 0 | 0 | 0 |
| Published/rs2308765 | 6 HLA-DRB1 | 32638316 | 0 | 0 | 0 |
| Published/rs1784195 | 6 HLA-DRB1 | 32574243 | 0 | 0 | 0 |
| Published/rs1784194 | 6 HLA-DRB1 | 32574238 | 0 | 0 | 0 |
| Published/rs1682297 | 6 HLA-DRB1 | 32574194 | 0 | 0 | 0 |
| Published/rs1682297 | 6 HLA-DRB1 | 32574143 | 0 | 0 | 0 |
| Published/rs1682288 | 6 HLA-DRB1 | 32549213 | 0 | 0 | 0 |
| Published/rs1682288 | 6 HLA-DRB1 | 32549212 | 0 | 0 | 0 |
| Published/rs1060346 | 6 HLA-DRB1 | 32640765 | 0 | 0 | 0 |
| Published/rs1059596 | 6 HLA-DRB1 | 32640687 | 0 | 0 | 0 |
| Published/rs707953 | 6 HLA-DRB1 | 32665484 | 0 | 0 | 0 |
| Published/rs701884 | 6 HLA-DRB1 | 32665457 | 0 | 0 | 0 |
| Published/rs3828815 | 6 HLA-DRB1 | 32656559 | 0 | 0 | 0 |
| Published/rs2308783 | 6 HLA-DRB1 | 32656528 | 0 | 0 | 0 |
| Published/rs2308777 | 6 HLA-DRB1 | 32657370 | 0 | 0 | 0 |
| Published/rs2308772 | 6 HLA-DRB1 | 32657421 | 0 | 0 | 0 |
| Published/rs2308770 | 6 HLA-DRB1 | 32657442 | 0 | 0 | 0 |
| Published/rs2308767 | 6 HLA-DRB1 | 32657475 | 0 | 0 | 0 |
| Published/rs2308764 | 6 HLA-DRB1 | 32657526 | 0 | 0 | 0 |
| Published/rs2308763 | 6 HLA-DRB1 | 32657541 | 0 | 0 | 0 |
| Published/rs2308762 | 6 HLA-DRB1 | 32657559 | 0 | 0 | 0 |
| Published/rs2308761 | 6 HLA-DRB1 | 32657565 | 0 | 0 | 0 |
| Published/rs2308760 | 6 HLA-DRB1 | 32657567 | 0 | 0 | 0 |
| Published/rs2308759 | 6 HLA-DRB1 | 32657574 | 0 | 0 | 0 |
| Published/rs2308758 | 6 HLA-DRB1 | 32657585 | 0 | 0 | 0 |
| Published/rs2308757 | 6 HLA-DRB1 | 32657589 | 0 | 0 | 0 |
| Published/rs2308756 | 6 HLA-DRB1 | 32657591 | 0 | 0 | 0 |
| Published/rs2308755 | 6 HLA-DRB1 | 32657592 | 0 | 0 | 0 |
| Published/rs1784195 | 6 HLA-DRB1 | 32657526 | 0 | 0 | 0 |
| Published/rs1784195 | 6 HLA-DRB1 | 32657475 | 0 | 0 | 0 |
| Published/rs1689789 | 6 HLA-DRB1 | 32657380 | 0 | 0 | 0 |
| Published/rs1682297 | 6 HLA-DRB1 | 32657433 | 0 | 0 | 0 |
| Published/rs1682297 | 6 HLA-DRB1 | 32657421 | 0 | 0 | 0 |
| Published/rs1682285 | 6 HLA-DRB1 | 32659935 | 0 | 0 | 0 |
| Published/rs1155446 | 6 HLA-DRB1 | 32659913 | 0 | 0 | 0 |
| Published/rs1064594 | 6 HLA-DRB1 | 32659931 | 0 | 0 | 0 |
| Published/rs1064587 | 6 HLA-DRB1 | 32665456 | 0 | 0 | 0 |
| Published/rs1059586 | 6 HLA-DRB1 | 32659968 | 0 | 0 | 0 |
| Published/rs1059582 | 6 HLA-DRB1 | 32659995 | 0 | 0 | 0 |
| Published/rs1059553 | 6 HLA-DRB1 | 32665461 | 0 | 0 | 0 |
| Published/rs1059551 | 6 HLA-DRB1 | 32665466 | 0 | 0 | 0 |
| Published/rs1059548 | 6 HLA-DRB1 | 32665482 | 0 | 0 | 0 |
| Published/rs9469203 | 6 HLA-DQA1 | 32713244 | 0 | 0 | 0 |
| Published/rs9272793 | 6 HLA-DQA1 | 32718473 | 0 | 0 | 0 |
| Published/rs9272789 | 6 HLA-DQA1 | 32718439 | 0 | 0 | 0 |
| Published/rs9272785 | 6 HLA-DQA1 | 32718379 | 0 | 0 | 0 |
| Published/rs9272745 | 6 HLA-DQA1 | 32717784 | 0 | 0 | 0 |
| Published/rs9272711 | 6 HLA-DQA1 | 32717290 | 0 | 0 | 0 |
| Published/rs9272709 | 6 HLA-DQA1 | 32717257 | 0 | 0 | 0 |
| Published/rs9272708 | 6 HLA-DQA1 | 32717256 | 0 | 0 | 0 |
| Published/rs9272706 | 6 HLA-DQA1 | 32717249 | 0 | 0 | 0 |
| Published/rs9272705 | 6 HLA-DQA1 | 32717242 | 0 | 0 | 0 |
| Published/rs9272704 | 6 HLA-DQA1 | 32717233 | 0 | 0 | 0 |
| Published/rs9272703 | 6 HLA-DQA1 | 32717232 | 0 | 0 | 0 |
| Published/rs9272700 | 6 HLA-DQA1 | 32717208 | 0 | 0 | 0 |
| Published/rs9272699 | 6 HLA-DQA1 | 32717207 | 0 | 0 | 0 |
| Published/rs9272698 | 6 HLA-DQA1 | 32717202 | 0 | 0 | 0 |
| Published/rs9272697 | 6 HLA-DQA1 | 32717201 | 0 | 0 | 0 |
| Published/rs9272696 | 6 HLA-DQA1 | 32717200 | 0 | 0 | 0 |
| Published/rs9272695 | 6 HLA-DQA1 | 32717194 | 0 | 0 | 0 |
| Published/rs9272693 | 6 HLA-DQA1 | 32717190 | 0 | 0 | 0 |
| Published/rs9272692 | 6 HLA-DQA1 | 32717185 | 0 | 0 | 0 |
| Published/rs9272691 | 6 HLA-DQA1 | 32717170 | 0 | 0 | 0 |
| Published/rs9272689 | 6 HLA-DQA1 | 32717083 | 0 | 0 | 0 |
| Published/rs9272430 | 6 HLA-DQA1 | 32713235 | 0 | 0 | 0 |
| Published/rs7990 | 6 HLA-DQA1 | 32717943 | 0 | 0 | 0 |
| Published/rs707963 | 6 HLA-DQA1 | 32717947 | 0 | 0 | 0 |
| Published/rs707962 | 6 HLA-DQA1 | 32717952 | 0 | 0 | 0 |
| Published/rs707952 | 6 HLA-DQA1 | 32717784 | 0 | 0 | 0 |
| Published/rs707950 | 6 HLA-DQA1 | 32717851 | 0 | 0 | 0 |
| Published/rs707949 | 6 HLA-DQA1 | 32717930 | 0 | 0 | 0 |
| Published/rs4193 | 6 HLA-DQA1 | 32717214 | 0 | 0 | 0 |
| Published/rs2308891 | 6 HLA-DQA1 | 32717987 | 0 | 0 | 0 |
| Published/rs2308885 | 6 HLA-DQA1 | 32717942 | 0 | 0 | 0 |
| Published/rs2308883 | 6 HLA-DQA1 | 32717852 | 0 | 0 | 0 |
| Published/rs1272209 | 6 HLA-DQA1 | 32718415 | 0 | 0 | 0 |
| Published/rs1272209 | 6 HLA-DQA1 | 32717877 | 0 | 0 | 0 |
| Published/rs1272208 | 6 HLA-DQA1 | 32717293 | 0 | 0 | 0 |
| Published/rs1272208 | 6 HLA-DQA1 | 32717280 | 0 | 0 | 0 |
| Published/rs1272208 | 6 HLA-DQA1 | 32717279 | 0 | 0 | 0 |
| Published/rs1272207 | 6 HLA-DQA1 | 32717245 | 0 | 0 | 0 |
| Published/rs1272207 | 6 HLA-DQA1 | 32717236 | 0 | 0 | 0 |
| Published/rs1272207 | 6 HLA-DQA1 | 32717222 | 0 | 0 | 0 |
| Published/rs1272207 | 6 HLA-DQA1 | 32717218 | 0 | 0 | 0 |
| Published/rs1272207 | 6 HLA-DQA1 | 32717211 | 0 | 0 | 0 |
| Published/rs1272206 | 6 HLA-DQA1 | 32717205 | 0 | 0 | 0 |
| Published/rs1272206 | 6 HLA-DQA1 | 32717201 | 0 | 0 | 0 |
| Published/rs1272206 | 6 HLA-DQA1 | 32717199 | 0 | 0 | 0 |
| Published/rs1272205 | 6 HLA-DQA1 | 32717191 | 0 | 0 | 0 |
| Published/rs1272205 | 6 HLA-DQA1 | 32717190 | 0 | 0 | 0 |
| Published/rs1272205 | 6 HLA-DQA1 | 32717181 | 0 | 0 | 0 |
| Published/rs1272205 | 6 HLA-DQA1 | 32717173 | 0 | 0 | 0 |
| Published/rs1272205 | 6 HLA-DQA1 | 32717125 | 0 | 0 | 0 |
| Published/rs1272204 | 6 HLA-DQA1 | 32717113 | 0 | 0 | 0 |
| Published/rs1272204 | 6 HLA-DQA1 | 32713287 | 0 | 0 | 0 |
| Published/rs1272204 | 6 HLA-DQA1 | 32713266 | 0 | 0 | 0 |
| Published/rs1272203 | 6 HLA-DQA1 | 32713262 | 0 | 0 | 0 |
| Published/rs1129749 | 6 HLA-DQA1 | 32717106 | 0 | 0 | 0 |
| Published/rs1128744 | 6 HLA-DQA1 | 32717096 | 0 | 0 | 0 |
| Published/rs1071630 | 6 HLA-DQA1 | 32717104 | 0 | 0 | 0 |
| Published/rs1048430 | 6 HLA-DQA1 | 32718465 | 0 | 0 | 0 |
| Published/rs1048090 | 6 HLA-DQA1 | 32717271 | 0 | 0 | 0 |
| Published/rs1048089 | 6 HLA-DQA1 | 32717266 | 0 | 0 | 0 |
| Published/rs1048063 | 6 HLA-DQA1 | 32717217 | 0 | 0 | 0 |
| Published/rs1048052 | 6 HLA-DQA1 | 32717209 | 0 | 0 | 0 |
| Published/rs9272794 | 6 HLA-DQA1 | 32718513 | 0 | 0 | 0 |
| Published/rs9272789 | 6 HLA-DQA1 | 32718414 | 0 | 0 | 0 |
| Published/rs9272786 | 6 HLA-DQA1 | 32718381 | 0 | 0 | 0 |
| Published/rs9272746 | 6 HLA-DQA1 | 32717791 | 0 | 0 | 0 |
| Published/rs9272709 | 6 HLA-DQA1 | 32717255 | 0 | 0 | 0 |
| Published/rs9272702 | 6 HLA-DQA1 | 32717231 | 0 | 0 | 0 |
| Published/rs9272694 | 6 HLA-DQA1 | 32717192 | 0 | 0 | 0 |
| Published/rs9272688 | 6 HLA-DQA1 | 32717075 | 0 | 0 | 0 |
| Published/rs9272433 | 6 HLA-DQA1 | 32713273 | 0 | 0 | 0 |
| Published/rs9272432 | 6 HLA-DQA1 | 32713252 | 0 | 0 | 0 |
| Published/rs9272431 | 6 HLA-DQA1 | 32713249 | 0 | 0 | 0 |
| Published/rs707951 | 6 HLA-DQA1 | 32717791 | 0 | 0 | 0 |
| Published/rs2308890 | 6 HLA-DQA1 | 32821805 | 0 | 0 | 0 |
| Published/rs2308889 | 6 HLA-DQA1 | 32821799 | 0 | 0 | 0 |
| Published/rs12722089 | 6 HLA-DQA1 | 32717303 | 0 | 0 | 0 |
| Published/rs12722084 | 6 HLA-DQA1 | 32717267 | 0 | 0 | 0 |
| Published/rs12722080 | 6 HLA-DQA1 | 32717252 | 0 | 0 | 0 |
| Published/rs12722050 | 6 HLA-DQA1 | 32717120 | 0 | 0 | 0 |
| Published/rs12722040 | 6 HLA-DQA1 | 32717084 | 0 | 0 | 0 |
| Published/rs12722043 | 6 HLA-DQA1 | 32717072 | 0 | 0 | 0 |
| Published/rs1129753 | 6 HLA-DQA1 | 32820957 | 0 | 0 | 0 |
| Published/rs1048419 | 6 HLA-DQA1 | 32718459 | 0 | 0 | 0 |
| Published/rs1048414 | 6 HLA-DQA1 | 32718456 | 0 | 0 | 0 |
| Published/rs1048381 | 6 HLA-DQA1 | 32822053 | 0 | 0 | 0 |
| Published/rs1048173 | 6 HLA-DQA1 | 32717833 | 0 | 0 | 0 |
| Published/rs1048134 | 6 HLA-DQA1 | 32717767 | 0 | 0 | 0 |
| Published/rs1048124 | 6 HLA-DQA1 | 32717761 | 0 | 0 | 0 |
| Published/rs1048087 | 6 HLA-DQA1 | 32717264 | 0 | 0 | 0 |
| Published/rs1048027 | 6 HLA-DQA1 | 32717147 | 0 | 0 | 0 |
| Published/rs1801284 | 19 HA1 | 101 9738 | 0 | 0 | 0 |
| Published/rs4630 | 22 GSTT1 | 22700876 | 0 | 0 | 0 |
| Published/rs2266637 | 22 GSTT1 | 22701 399 | 0 | 0 | 0 |
| Published/rs2266633 | 22 GSTT1 | 22701483 | 0 | 0 | 0 |
| Published/rs2234953 | 22 GSTT1 | 22701387 | 0 | 0 | 0 |
| Published/rs947894 | 11 GSTP1 | 67109265 | 0 | 0 | 0 |
| Published/rs8191449 | 11 GSTP1 | 67108957 | 0 | 0 | 0 |
| Published/rs749174 | 11 GSTP1 | 67109829 | 0 | 0 | 0 |
| Published/rs4986948 | 11 GSTP1 | 67109281 | 0 | 0 | 0 |
| Published/rs1065411 | 1 GSTM1 | 1.1E+08 | 0 | 0 | 0 |
| Published/rs756627 | 1 GSTM1 | 1.1E+08 | 0 | 0 | 0 |
| Published/rs2071487 | 1 GSTM1 | 1.1E+08 | 0 | 0 | 0 |
| Published/rs1110198 | 1 GSTM1 | 1.1E+08 | 0 | 0 | 0 |
| Published/rs945635 | 1 FCRL3 | 1.54E+08 | 0 | 0 | 0 |
| Published/rs7528684 | 1 FCRL3 | 1.54E+08 | 0 | 0 | 0 |
| Published/rs3761959 | 1 FCRL3 | 1.54E+08 | 0 | 0 | 0 |
| Published/rs1126479 | 1 FCRL3 | 1.54E+08 | 0 | 0 | 0 |
| Published/rs763110 | 1 faslg | 1.64E+08 | 0 | 0 | 0 |
| Published/rs3218621 | 10 fas | 90752876 | 0 | 0 | 0 |
| Published/rs2234978 | 10 fas | 9076 1809 | 0 | 0 | 0 |
| Published/rs2234767 | 10 fas | 90739236 | 0 | 0 | 0 |
| Published/rs1800682 | 10 fas | 90739943 | 0 | 0 | 0 |
| Published/rs9340799 | 6 ESR1ESR | 1.52E+08 | 0 | 0 | 0 |
| Published/rs2234693 | 6 ESR1ESR | 1.52E+08 | 0 | 0 | 0 |
| Published/rs8179176 | 6 ESR1 | 1.52E+08 | 0 | 0 | 0 |
| Published/rs1115581 | 6 ESR1 | 1.52E+08 | 0 | 0 | 0 |
| Published/rs1115581 | 6 ESR1 | 1.52E+08 | 0 | 0 | 0 |
| Published/rs887826 | 7 EGFR | 54928937 | 0 | 0 | 0 |
| Published/rs718836 | 7 EGFR | 55016513 | 0 | 0 | 0 |
| Published/rs4947487 | 7 EGFR | 54848995 | 0 | 0 | 0 |
| Published/rs17172429 | 7 EGFR | 54889367 | 0 | 0 | 0 |
| Published/rs1323958 | 7 EGFR | 54815866 | 0 | 0 | 0 |
| Published/rs1153663 | 7 EGFR | 55003224 | 0 | 0 | 0 |
| Published/rs1005176 | EGFR | 5481 1106 | 0 | 0 | 0 |
| Published/rs1053874 | 16 DNasel | 3647748 | 0 | 0 | 0 |
| Published/rs179982 | 6 DNasel | 16461117 | 0 | 0 | 0 |
| Published/rs1059857 | 16 DNasel | 3648194 | 0 | 0 | 0 |
| Published/rs1030874 | 2 DNasel | 2.12E+08 | 0 | 0 | 0 |
| Published/rs743572 | 10 cyp17a1 | 1.05E+08 | 0 | 0 | 0 |
| Published/rs733618 | 2 CTLA4CTL | 2.05E+08 | 0 | 0 | 0 |
| Published/rs5742909 | 2 CTLA4CTL | 2.05E+08 | 0 | 0 | 0 |
| Published/rs231775 | 2 CTLA4 | 2.05E+08 | 0 | 0 | 0 |
| Published/rs11571311 | 2 CTLA4 | 2.05E+08 | 0 | 0 | 0 |
| Published/rs6691117 | 1 CR1 | 2.04E+08 | 0 | 0 | 0 |
| Published/rs4844609 | 1 CR1 | 2.04E+08 | 0 | 0 | 0 |
| Published/rs3991747 | 1 CR1 | 2.04E+08 | 0 | 0 | 0 |
| Published/rs3811381 | 1 CR1 | 2.04E+08 | 0 | 0 | 0 |
| Published/rs3737002 | 1 CR1 | 2.04E+08 | 0 | 0 | 0 |
| Published/rs2296160 | 1 CR1 | 2.04E+08 | 0 | 0 | 0 |
| Published/rs2274567 | 1 CR1 | 2.04E+08 | 0 | 0 | 0 |
| Published/rs1725904 | 1 CR1 | 2.04E+08 | 0 | 0 | 0 |
| Published/rs1704766 | 1 CR1 | 2.04E+08 | 0 | 0 | 0 |
| Published/rs1704766 | 1 CR1 | 2.04E+08 | 0 | 0 | 0 |
| Published/rs1158794 | 1 CR1 | 2.04E+08 | 0 | 0 | 0 |
| Published/rs1800561 | 4 CD38 | 15502827 | 0 | 0 | 0 |
| Published/rs6449182 | 4 CD38 | 15456722 | 0 | 0 | 0 |
| Published/rs2904880 | 16 CD19 | 28851897 | 0 | 0 | 0 |
| Published/rs1799864 | 3 ccr2 | 46374212 | 0 | 0 | 0 |
| Published/rs172378 | 1 C1QA | 22710744 | 0 | 0 | 0 |
| Published/rs1800477 | 18 bc12 | 59136753 | 0 | 0 | 0 |
| Published/rs8178847 | 17 APOH | 61647277 | 0 | 0 | 0 |
| Published/rs4581 | 17 APOH | 61641219 | 0 | 0 | 0 |
| Published/rs3826358 | 17 APOH | 61655946 | 0 | 0 | 0 |
| Published/rs3176975 | 17 APOH | 61641219 | 0 | 0 | 0 |
| Published/rs1803124 | 17 APOH | 61654612 | 0 | 0 | 0 |
| Published/rs1803122 | 17 APOH | 61647316 | 0 | 0 | 0 |
| Published/rs1801692 | 17 APOH | 61652626 | 0 | 0 | 0 |
| Published/rs1801690 | 17 APOH | 61638747 | 0 | 0 | 0 |
| Published/rs1801689 | 17 APOH | 61641042 | 0 | 0 | 0 |
| Published/rs12544 | 17 APOH | 61652674 | 0 | 0 | 0 |
| Published/rs1155196 | 17 APOH | 61647247 | 0 | 0 | 0 |
| Published/rs7412 | 19 APOE | 50103919 | 0 | 0 | 0 |
| Published/rs429358 | 19 APOE | 50103781 | 0 | 0 | 0 |
| Published/rs4366 | 17 ACE | 58929187 | 0 | 0 | 0 |
| Published/rs4343 | 17 ACE | 58919763 | 0 | 0 | 0 |
| Published/rs1799763 | 17 ACE | 58929190 | 0 | 0 | 0 |
| Published/rs1024611 | 17 CCL2 | 29603901 | 0 | 0 | 0 |
| Published/rs1061622 | 1 TNFRSF1B | 12187221 | 0 | 0 | 0 |
| Published/rs1100312 | 10 MBL2 | 54202020 | 0 | 0 | 0 |
| Published/rs1108582 | 19 DNASE2 | 12854255 | 0 | 0 | 0 |
| Published/rs1697219 | 13 TNFSF13B | 1.08E+08 | 0 | 0 | 0 |
| Published/rs1697219 | 13 TNFSF13B | 1.08E+08 | 0 | 0 | 0 |
| Published/rs1788031 | 1 TNFRSF1B | 12183208 | 0 | 0 | 0 |
| Published/rs1788343 | 1 TNFRSF1B | 12187221 | 0 | 0 | 0 |
| Published/rs1788684 | 1 TNFRSF1B | 12187328 | 0 | 0 | 0 |
| Published/rs2107538 | 17 CCL5 | 31231893 | 0 | 0 | 0 |
| Published/rs2227306 | 4 IL8 | 74972090 | 0 | 0 | 0 |
| Published/rs2227532 | 4 IL8 | 74970567 | 0 | 0 | 0 |
| Published/rs2275415 | 1 TNFRSF1B | 12186839 | 0 | 0 | 0 |
| Published/rs2278658 | 15 LTK | 39584263 | 0 | 0 | 0 |
| Published/rs2280788 | 17 CCL5 | 31231518 | 0 | 0 | 0 |
| Published/rs2293682 | 19 DNASE2 | 12850560 | 0 | 0 | 0 |
| Published/rs3759465 | 13 TNFSF13B | 1.08E+08 | 0 | 0 | 0 |
| Published/rs4073 | 4 IL8 | 74971059 | 0 | 0 | 0 |
| Published/rs419478 | 1 RA84A | 2.26E+08 | 0 | 0 | 0 |
| Published/rs4804209 | 19 DNASE2M | 12846955 | 0 | 0 | 0 |
| Published/rs5746026 | 1 TNFRSF1B | 12187328 | 0 | 0 | 0 |
| Published/rs699 | 1 AGT | 2.27E+08 | 0 | 0 | 0 |
| Published/rs7096206 | 10 MBL2 | 54201691 | 0 | 0 | 0 |
| Published/rs8179079 | 10 MBL2 | 54201300 | 0 | 0 | 0 |
| Published/rs9435830 | 1 RAB4A | 2.26E+08 | 0 | 0 | 0 |
| Published/rs945439 | 1 TNFRSF1B | 12183208 | 0 | 0 | 0 |
| Published/rs9514828 | 13 TNFSF13B | 1.08E+08 | 0 | 0 | 0 |

### III. Amplifying a Target Sequence

In a particular embodiment, it may be desirable to amplify the target sequence before evaluating the SNP. Nucleic acids used as a template for amplification may be isolated from cells, tissues or other samples according to standard methodologies (Sambrook et al., 1989). In certain embodiments, analysis is performed on whole cell or tissue homogenates or biological fluid samples without substantial purification of the template nucleic acid. The nucleic acid may be genomic DNA or fractionated or whole cell RNA. Where RNA is used, it may be desired to first convert the RNA to a complementary DNA. The DNA also may be from a cloned source or synthesized in vitro.

The term “primer,” as used herein, is meant to encompass any nucleic acid that is capable of priming the synthesis of a nascent nucleic acid in a template-dependent process. Typically, primers are oligonucleotides from ten to twenty or thirty base pairs in length, but longer sequences can be employed. Primers may be provided in double-stranded or single-stranded form, although the single-stranded form is preferred.

Pairs of primers designed to selectively hybridize to nucleic acids flanking the polymorphic site are contacted with the template nucleic acid under conditions that permit selective hybridization. Depending upon the desired application, high stringency hybridization conditions may be selected that will only allow hybridization to sequences that are completely complementary to the primers. In other embodiments, hybridization may occur under reduced stringency to allow for amplification of nucleic acids containing one or more mismatches with the primer sequences. Once hybridized, the template-primer complex is contacted with one or more enzymes that facilitate template-dependent nucleic acid synthesis. Multiple rounds of amplification, also referred to as “cycles,” are conducted until a sufficient amount of amplification product is produced.

It is also possible that multiple target sequences will be amplified in a single reaction. Primers designed to expand specific sequences located in different regions of the target genome, thereby identifying different polymorphisms, would be mixed together in a single reaction mixture. The resulting amplification mixture would contain multiple amplified regions, and could be used as the source template for polymorphism detection using the methods described in this application.

A number of template dependent processes are available to amplify the oligonucleotide sequences present in a given template sample. One of the best known amplification methods is the polymerase chain reaction (referred to as PCR™), which is described in detail in U.S. Pat. Nos. 4,683,195, 4,683,202 and 4,800,159, and in Innis et al., 1988, each of which is incorporated herein by reference in their entirety.

A reverse transcriptase PCR™ amplification procedure may be performed when the source of nucleic acid is fractionated or whole cell RNA. Methods of reverse transcribing RNA into cDNA are well known (see Sambrook et al., 1989). Alternative methods for reverse polymerization utilize thermostable DNA polymerases. These methods are described in WO 90/07641. Polymerase chain reaction methodologies are well known in the art. Representative methods of RT-PCR are described in U.S. Pat. No. 5,882,864.

Another method for amplification is ligase chain reaction (“LCR”), disclosed in European Application No. 320 308, incorporated herein by reference in its entirety. U.S. Pat. No. 4,883,750 describes a method similar to LCR for binding probe pairs to a target sequence. A method based on PCR™ and oligonucleotide ligase assay (OLA), disclosed in U.S. Pat. No. 5,912,148, may also be used.

Another ligase-mediated reaction is disclosed by Guilfoyle et al. (1997). Genomic DNA is digested with a restriction enzyme and universal linkers are then ligated onto the restriction fragments. Primers to the universal linker sequence are then used in PCR to amplify the restriction fragments. By varying the conditions of the PCR, one can specifically amplify fragments of a certain size (i.e., less than a 1000 bases). An example for use with the present invention would be to digest genomic DNA with XbaI, and ligate on MI 3-universal primers with an XbaI over hang, followed by amplification of the genomic DNA with an M13 universal primer. Only a small percentage of the total DNA would be amplified (the restriction fragments that were less than 1000 bases). One would then use labeled primers that correspond to a SNP are located within XbaI restriction fragments of a certain size (<1000 bases) to perform the assay. The benefit to using this approach is that each individual region would not have to be amplified separately. There would be the potential to screen thousands of SNPs from the single PCR reaction, i.e., multiplex potential.

Alternative methods for amplification of target nucleic acid sequences that may be used in the practice of the present invention are disclosed in U.S. Pat. Nos. 5,843,650, 5,846,709, 5,846,783, 5,849,546, 5,849,497, 5,849,547, 5,858,652, 5,866,366, 5,916,776, 5,922,574, 5,928,905, 5,928,906, 5,932,451, 5,935,825, 5,939,291 and 5,942,391, GB Application No. 2 202 328, and in PCT Application No. PCT/US89/01025, each of which is incorporated herein by reference in its entirety.

Qbeta Replicase, described in PCT Application No. PCT/US87/00880, may also be used as an amplification method in the present invention. In this method, a replicative sequence of RNA that has a region complementary to that of a target is added to a sample in the presence of an RNA polymerase. The polymerase will copy the replicative sequence, which may then be detected.

An isothermal amplification method, in which restriction endonucleases and ligases are used to achieve the amplification of target molecules that contain nucleotide 5′-[alpha-thio]-triphosphates in one strand of a restriction site may also be useful in the amplification of nucleic acids in the present invention (Walker et al., 1992). Strand Displacement Amplification (SDA), disclosed in U.S. Pat. No. 5,916,779, is another method of carrying out isothermal amplification of nucleic acids which involves multiple rounds of strand displacement and synthesis, i.e., nick translation.

Other nucleic acid amplification procedures include polymerization-based amplification systems (TAS), including nucleic acid sequence based amplification (NASBA) and 3SR (Kwoh et al., 1989; Gingeras et al., PCT Application WO 88/10315, incorporated herein by reference in their entirety). European Application No. 329 822 discloses a nucleic acid amplification process involving cyclically synthesizing single-stranded RNA (ssRNA), ssDNA, and double-stranded DNA (dsDNA), which may be used in accordance with the present invention.

PCT Application WO 89/06700 (incorporated herein by reference in its entirety) discloses a nucleic acid sequence amplification scheme based on the hybridization of a promoter region/primer sequence to a target single-stranded DNA (ssDNA) followed by polymerization of many RNA copies of the sequence. This scheme is not cyclic, i.e., new templates are not produced from the resultant RNA transcripts. Other amplification methods include “race” and “one-sided PCR” (Frohman, 1990; Ohara et al., 1989).

Another advantageous step is to prevent unincorporated NTPs from being incorporated in a subsequent primer extension reaction. Commercially available kits may be used to remove unincorporated NTPs from the amplification products. The use of shrimp alkaline phosphatase to destroy unincorporated NTPs is also a well-known strategy for this purpose.

### IV. Kits

All the essential materials and reagents required for detecting nucleic acid mutations in a sample may be assembled together in a kit. This generally will comprise a primer or probe designed to hybridize specifically to or upstream of target nucleotides of the polymorphism of interest. The primer or probe may be labeled with a radioisotope, a fluorophore, a chromophore, a dye, an enzyme, or TOF carrier. Also included may be enzymes suitable for amplifying nucleic acids, including various polymerases (reverse transcriptase, Taq, etc.), dNTPs/rNTPs and buffers (e.g., 10× buffer=100 mM Tris-HCl (pH 8.3), and 500 mM KCl) to provide the necessary reaction mixture for amplification. One or more of the deoxynucleotides may be labeled with a radioisotope, a fluorophore, a chromophore, a dye, or an enzyme. Such kits may also include enzymes and other reagents suitable for detection of specific nucleic acids or amplification products.

The container means of the kits will generally include at least one vial, test tube, flask, bottle, or other container means, into which a component may be placed, and preferably, suitably aliquoted. Where there is more than one component in the kit, the kit also will generally contain additional containers into which the additional components may be separately placed. However, various combinations of components may be comprised in a container. The kits of the present invention also will typically include a means for packaging the component containers in close confinement for commercial sale. Such packaging may include injection or blow-molded plastic containers into which the desired component containers are retained.

### V. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

All of the SNPs identified in Tables X and Z are candidates screened in a large genetic association study using SLE patients and control samples. Using existing hybridization technologies for SNP assays, groups of 1581-1800 SNPs will be assayed for genetic association with the development of SLE and other autoimmune diseases or associated sub-phenotypes such as clinical symptoms or outcomes of traditional clinical testing. Associated SNPs will be added to the list of SNPs useful as markers for diagnosis of the relevant disease.

The SNPs in Tables X and Z will be arrayed using a custom bead-based system from Illumina (San Diego, Calif.). Their systems can accommodate throughput ranging from several thousand to well over one million genotypes per day. Examples of useful products include the Illumina BeadStation 500G and BeadLab. These products permit SNP genotyping assays processed in an automated, production-scale environment.

### Example 2

A genetic association study was performed by genotyping four single nucleotide polymorphisms (SNPs) in the IL-21 gene in a total of 2636 samples (1318 cases and 1318 controls matched for age, sex and race). Genotyping was performed on the Illumina BeadStation 500GX system at the University of Texas Southwestern Microarray Core Facility (Dallas, Tex.). Population-based case-control association designs were employed.

A genetic association with SLE and two SNPs located within the second intron of IL-21 (rs907715: chi²=11.55, p=0.00068; rs2221903: chi²=5.49, p=0.019) was demonstrated. Upon stratification by race, the genetic association observed with both SNPs appears to arise from the European-American lupus patients. Furthermore, genotypes homozygous for the risk alleles were more frequent than genotypes homozygous for the non-risk alleles in European-American patients as compared to controls (rs907715 (GG versus AA):odds ratio=1.66, p=0.0049; rs2221903 (GG versus AA):Odds ratio=1.60, p=0.025). Lupus patients homozygous for the risk allele in either of the associated SNPs are as twice as likely to suffer from photosensitivity compared to patients homozygous for the non-risk allele (rs907715: chi²=9.69, p=0.0019; rs2221903: chi²=7.07, p=0.0078).

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope, and concept of the invention as defined by the appended claims.

### VI. References

The following references, to the extent that they provide exemplary procedural or other details supplementary to those set forth herein, are specifically incorporated herein by reference.
U.S. Pat. No. 4,659,774
U.S. Pat. No. 4,683,195
U.S. Pat. No. 4,683,202
U.S. Pat. No. 4,800,159
U.S. Pat. No. 4,816,571
U.S. Pat. No. 4,883,750
U.S. Pat. No. 4,959,463
U.S. Pat. No. 5,141,813
U.S. Pat. No. 5,242,974
U.S. Pat. No. 5,264,566
U.S. Pat. No. 5,279,271
U.S. Pat. No. 5,384,261
U.S. Pat. No. 5,405,783
U.S. Pat. No. 5,412,087
U.S. Pat. No. 5,424,186
U.S. Pat. No. 5,428,148
U.S. Pat. No. 5,429,807
U.S. Pat. No. 5,436,327
U.S. Pat. No. 5,445,934
U.S. Pat. No. 5,472,672
U.S. Pat. No. 5,527,681
U.S. Pat. No. 5,529,756
U.S. Pat. No. 5,532,128
U.S. Pat. No. 5,545,531
U.S. Pat. No. 5,554,501
U.S. Pat. No. 5,554,744
U.S. Pat. No. 5,556,752
U.S. Pat. No. 5,561,071
U.S. Pat. No. 5,571,639
U.S. Pat. No. 5,574,146
U.S. Pat. No. 5,593,839
U.S. Pat. No. 5,599,695
U.S. Pat. No. 5,602,244
U.S. Pat. No. 5,624,711
U.S. Pat. No. 5,658,734
U.S. Pat. No. 5,700,637
U.S. Pat. No. 5,757,994
U.S. Pat. No. 5,788,166
U.S. Pat. No. 5,838,002
U.S. Pat. No. 5,840,873
U.S. Pat. No. 5,843,640
U.S. Pat. No. 5,843,650
U.S. Pat. No. 5,843,651
U.S. Pat. No. 5,843,663
U.S. Pat. No. 5,846,708
U.S. Pat. No. 5,846,709
U.S. Pat. No. 5,846,717
U.S. Pat. No. 5,846,726
U.S. Pat. No. 5,846,729
U.S. Pat. No. 5,846,783
U.S. Pat. No. 5,849,481
U.S. Pat. No. 5,849,486
U.S. Pat. No. 5,849,487
U.S. Pat. No. 5,849,497
U.S. Pat. No. 5,849,546
U.S. Pat. No. 5,849,547
U.S. Pat. No. 5,851,772
U.S. Pat. No. 5,853,990
U.S. Pat. No. 5,853,992
U.S. Pat. No. 5,853,993
U.S. Pat. No. 5,856,092
U.S. Pat. No. 5,858,652
U.S. Pat. No. 5,861,244
U.S. Pat. No. 5,863,732
U.S. Pat. No. 5,863,753
U.S. Pat. No. 5,866,331
U.S. Pat. No. 5,866,366
U.S. Pat. No. 5,882,864
U.S. Pat. No. 5,882,864
U.S. Pat. No. 5,900,481
U.S. Pat. No. 5,905,024
U.S. Pat. No. 5,910,407
U.S. Pat. No. 5,912,124
U.S. Pat. No. 5,912,145
U.S. Pat. No. 5,912,148
U.S. Pat. No. 5,916,776
U.S. Pat. No. 5,916,779
U.S. Pat. No. 5,919,626
U.S. Pat. No. 5,919,630
U.S. Pat. No. 5,922,574
U.S. Pat. No. 5,925,517
U.S. Pat. No. 5,928,862
U.S. Pat. No. 5,928,869
U.S. Pat. No. 5,928,905
U.S. Pat. No. 5,928,906
U.S. Pat. No. 5,929,227
U.S. Pat. No. 5,932,413
U.S. Pat. No. 5,932,451
U.S. Pat. No. 5,935,791
U.S. Pat. No. 5,935,825
U.S. Pat. No. 5,939,291
U.S. Pat. No. 5,942,391
U.S. Pat. No. 5,986,258
U.S. Pat. No. 6,004,755
U.S. Pat. RE 35,413
U.S. Ser. No. 10/407,846
Arend and Dayer, Arthritis Rheum., 38:151-160, 1995.
Arend et al., Annu. Rev. Immunol., 16:27-55, 1998.
Arnett et al., Rheumatic Diseases Clinics of North America, 18:865-92, 1992.
Baechler et al., Proc. Natl. Acad. Sci. USA, 100(5):2610-15, 2003.
Bahr et al., J Mass Spectrom., 32:1111-1116, 1997.
Beaucage, Methods Mol. Biol., 20:33-61, 1993.
Bentzley et al., Anal Chem., 68(13):2141-2146, 1996.
Bucknall et al., J. Am. Soc. Mass Spectrom., 13(9):1015-1027, 2002.
Burger and Dayer, Neurology, 45(6S-6):S39-43, 1995.
Caprioli et al., Anal. Chem., 69:4751, 1997.
Chaurand et al., Anal Chem., 71(23):5263-5270, 1999.
Chen et al., Nat. Biotechnol., 19:537-542, 2001.
Desiderio et al., J. Mass Spectrom., 35(6):725-733, 2000.
Desiderio et al., Methods Mol. Biol., 61:57-65, 1996.
Dinarello, Int. Rev. Immunol., 16:457-499, 1998.
Duncan et al., Rapid Commun. Mass Spectrom., 7(12):1090-1094, 1993.
Eastgate et al., Lancet, 2:706-709, 1988.
European Appln No. 320 308
European Appln. No. 329 822
Faulstich et al., Anal. Chem., 69(21):4349-4353, 1997.
Fenn et al., Science, 246(4926):64-71, 1989.
Firestein et al., Arthritis Rheum., 37:644-652, 1994.
Frohman, In: PCR Protocols: A Guide To Methods And Applications, Academic Press, N.Y., 1990.
Fujikawa et al., Ann. Rheum. Dis., 54:318-320, 1995.
Gaffney et al., Proc. Natl. Acad. Sci. USA, 95: 14875-79, 1998.
GB Appln. No. 2 202 328
Gillam et al., J. Biol. Chem., 253(8):2532-2539, 1978.
Gobom et al., Anal. Chem., 72(14):3320-3326, 2000.
Guilfoyle et al., Nucleic Acids Research, 25:1854-1858, 1997.
Hannum et al., Nature, 343:336-340, 1990.
Harley et al., Current Opinions in Immunology, 10:690-96, 1998.
Horak et al., Rapid Commun. Mass Spectrom., 15(4):241-248, 2001.
Innis et al., Proc. Natl. Acad. Sci. USA, 85(24):9436-9440, 1988.
Itakura et al., J. Am. Chem. Soc., 97(25):7327-7332, 1975.
Jespersen et al., Anal Chem., 71(3):660-666, 1999.
Jiang et al., J. Agric. Food Chem., 48:3305, 2000.
Jonsson and Brokstad, In: A Textbook of Rheumatology, 6^{th} Ed., Philadelphia, Lippincott Williams & Wilkins, 495-504, 2001.
Jonsson et al., Oral Dis., 8:130-140, 2002.
Kabarle et al., Anal. Chem. 65(20):972A-986A, 1993.
Kahle et al., Ann. Rheum. Dis., 51:731-734, 1992.
Kanazawa et al., Biol. Pharm. Bull., 22(4):339-346, 1999.
Kazmaier et al., Anesthesiology, 89(4):831-817, 1998.
Khorana, Science, 203(4381):614-625, 1979.
Kuehn, JAMA, 293:1315, 2005.
Kwoh et al., Proc. Natl. Acad. Sci. USA, 86: 1173, 1989.
Li et al., Trends Biotechnol., 18:151, 2000.
Lipsky, In: Harrison's principles of internal medicine, Fauci et al. (Eds.), 14^{th} Ed., NY, McGraw-Hill, 1880-1888, 1998.
Lovelace et al., J. Chromatogr., 562(1-2):573-584, 1991.
Lynn et al., J. Mol. Evol., 48(5):605-614, 1999.
Marie et al., Anal. Chem., 72(20):5106-5114, 2000.
Miketova et al., Mol. Biotechnol., 8(3):249-253, 1997.
Mirgorodskaya et al., Rapid Commun. Mass Spectrom., 14(14):1226-1232, 2000.
Moser et al., Proc. Natl. Acad. Sci. USA, 95:14869-74, 1998.
Muddiman et al., Fres. J. Anal. Chem., 354:103, 1996.
Nelson et al., Anal. Chem., 66:1408, 1994.
Nguyen et al., J. Chromatogr. A., 705(1):21-45, 1995.
Ohara et al., Proc. Natl. Acad. Sci. USA, 86: 5673-5677, 1989.
PCT Appln. No. PCT/US87/00880
PCT Appln. No. PCT/US89/01025
PCT Appln. No. WO 88/10315
PCT Appln. No. WO 89/06700
PCT Appln. WO 90/07641
Prieur et al., Lancet., 2:1240-1242, 1987.
Roepstorff, EXS., 88:81-97, 2000.
Rooney et al., Rheumatol. Int., 10:217-219, 1990.
Salomonsson et al., Arthritis Rheum., 48:3187-201, 2003.
Salomonsson et al., Scand J. Immunol., 55: 336-342, 2002.
Sambrook et al., In: Molecular cloning, Cold Spring Harbor Laboratory Press, Cold Spring Harbor, N.Y., 1989.
Stoeckli et al., Nat. Med., 7(4):493-496, 2001.
Takach et al., J. Protein Chem., 16:363, 1997.
van den Berg, Semin. Arthritis Rheum., 30(5S-2):7-16, 2001.
Villanueva et al., Enzyme Microb. Technol., 29:99, 1999.
Wakeland et al., Immunity, 15:690-96, 2001.
Walker et al., Proc. Natl. Acad. Sci. USA, 89:392-396 1992.
Wang et al., Anal. Chem., 72(21):5285-5289, 2000.
Wang et al., J. Agric. Food. Chem., 47:1549, 1999.
Wang et al., J. Agric. Food. Chem., 47:2009, 1999.
Weyand and Goronzy, Ann. NY Acad. Sci., 987:140-149, 2003.
Wittmann et al., Biotechnol. Bioeng., 72:642, 2001.
Wu et al., Anal. Chem., 70:456A, 1998.
Wu et al., Biochem. Biophys. Res. Commun., 233(1):221-226, 1997.
Wu et al., Biochim. Biophys. Acta, 1466:315-327, 2000.
Xanthou et al., Arthritis Rheum., 44:408-418, 2001.
Yang et al., J. Agric. Food. Chem., 48:3990, 2000.
Zhong et al., Clin. Chem. ACTA., 313:147, 2001.
Zweigenbaum et al., Anal. Chem., 71(13):2294-300, 1999.
Zweigenbaum et al., J. Pharm. Biomed. Anal., 23(4):723-733, 2000.

What is claimed is:

1. A method of identifying a subject afflicted with or at risk of developing an autoimmune disease comprising:
(a) obtaining a nucleic acid-containing sample from said subject;
(b) analyzing a single nucleotide polymorphism (SNP) selected from those listed in Table X,
wherein the presence of a SNP from Table X indicates that said subject is afflicted or at risk of developing an autoimmune disease.

2. The method of claim 1, further comprising analyzing a second SNP from Table X.

3. The method of claim 2, further comprising analyzing a third SNP from Table X.

4. The method of claim 3, further comprising analyzing a fourth SNP from Table X.

5. The method of claim 4, further comprising analyzing a fifth SNP from Table X.

6. The method of claim 1, further comprising analyzing a SNP from Table Z.

7. The method of claim 6, further comprising analyzing a second, third, fourth or fifth SNP from Table Z.

8. The method of claim 1, wherein said autoimmune disease is systemic lupus erythematosus, Sjogren's syndrome, rheumatoid arthritis, juvenile onset diabetes mellitus, Wegener's granulomatosis, inflammatory bowel disease, polymyositis, dermatomyositis, multiple endocrine failure, Schmidt's syndrome, autoimmune uveitis, Addison's disease, adrenalitis, Graves' disease, thyroiditis, Hashimoto's thyroiditis, autoimmune thyroid disease, pernicious anemia, gastric atrophy, chronic hepatitis, lupoid hepatitis, atherosclerosis, presenile dementia, demyelinating diseases, multiple sclerosis, subacute cutaneous lupus erythematosus, hypoparathyroidism, Dressler's syndrome, myasthenia gravis, autoimmune thrombocytopenia, idiopathic thrombocytopenic purpura, hemolytic anemia, pemphigus vulgaris, pemphigus, dermatitis herpetiformis, alopecia arcata, pemphigoid, scleroderma, progressive systemic sclerosis, CREST syndrome (calcinosis, Raynaud's phenomenon, esophageal dysmotility, sclerodactyly, and telangiectasia), adult onset diabetes mellitus (Type II diabetes), male and female autoimmune infertility, ankylosing spondolytis, ulcerative colitis, Crohn's disease, mixed connective tissue disease, polyarteritis nedosa, systemic necrotizing vasculitis, juvenile onset rheumatoid arthritis, glomerulonephritis, atopic dermatitis, atopic rhinitis, Goodpasture's syndrome, Chagas' disease, sarcoidosis, rheumatic fever, asthma, recurrent abortion, anti-phospholipid syndrome, farmer's lung, erythema multiforme, post cardiotomy syndrome, Cushing's syndrome, autoimmune chronic active hepatitis, bird-fancier's lung, allergic disease, allergic encephalomyelitis, toxic epidermal necrolysis, alopecia, Alport's syndrome, alveolitis, allergic alveolitis, fibrosing alveolitis, interstitial lung disease, erythema nodosum, pyoderma gangrenosum, transfusion reaction, leprosy, malaria, leishmaniasis, trypanosomiasis, Takayasu's arteritis, polymyalgia rheumatica, temporal arteritis, schistosomiasis, giant cell arteritis, ascariasis, aspergillosis, Sampter's syndrome, eczema, lymphomatoid granulomatosis, Behcet's disease, Caplan's syndrome, Kawasaki's disease, dengue, encephalomyelitis, endocarditis, endomyocardial fibrosis, endophthalmitis, erythema elevatum et diutinum, psoriasis, erythroblastosis fetalis, eosinophilic faciitis, Shulman's syndrome, Felty's syndrome, filariasis, cyclitis, chronic cyclitis, heterochronic cyclitis, Fuch's cyclitis, IgA nephropathy, Henoch-Schonlein purpura, glomerulonephritis, graft versus host disease, transplantation rejection, human immunodeficiency virus infection, echovirus infection, cardiomyopathy, Alzheimer's disease, parvovirus infection, rubella virus infection, post vaccination syndromes, congenital rubella infection, Hodgkin's and Non-Hodgkin's lymphoma, renal cell carcinoma, multiple myeloma, Eaton-Lambert syndrome, relapsing polychondritis, malignant melanoma, cryoglobulinemia, Waldenstrom's macroglobulemia, Epstein-Barr virus infection, mumps, Evan's syndrome, and autoimmune gonadal failure.

9. The method of claim 1, further comprising treating said subject based on the results of step (b).

10. The method of claim 1, further comprising taking a clinical history from said subject.

11. The method of claim 1, wherein analysis comprises nucleic acid amplification.

12. The method of claim 12, wherein amplification comprises PCR.

13. The method of claim 1, wherein analysis comprises primer extension.

14. The method of claim 1, wherein analysis comprises restriction digestion.

15. The method of claim 1, wherein analysis comprises sequencing.

16. The method of claim 1, wherein analysis comprises SNP specific oligonucleotide hybridization.

17. The method of claim 1, wherein analysis comprises a DNAse protection assay.

18. The method of claim 1, wherein said sample is blood, sputum, saliva, mucosal scraping or tissue biopsy.
